(19) United States
(12) Patent Application Publication
(10) Pub. No.: US 2007/0207554 A1
(43) Pub. Date: Sep. 6, 2007

(54) Medical System and Method for Determining Parameters

(76) Inventors: Alex Chun Ching Lin, Cincinnati, OH (US); Wei-Kuo Chang, Taipei City (TW); Stanley Chang, Taipei City (TW)

Correspondence Address: WHYTE HIRSCHBOECK DUDEK S C, 555 EAST WELLS STREET, SUITE 1900, MILWAUKEE, WI, 53202, US

(21) Appl. No.: 11/735,173

(22) Filed: Apr. 13, 2007

Related U.S. Application Data
(63) Continuation-in-part of application No. 10/787,705, filed on Feb. 26, 2004.
(60) Provisional application No. 60/791,454, filed on Apr. 13, 2006.

(51) Int. Cl. G01N 33/558

(52) U.S. Cl. 436/514

(57) ABSTRACT

The present invention discloses a device or system, and the method thereof, for determining gastrointestinal parameters with the reference database saved equations for calculating the parameters comprising concentration of dietary formula, the gastric residual volume, the amount of dietary formula retained in the stomach, and volume of dietary formula remaining in stomach.

### RELATED APPLICATIONS

This application claims the priority of provisional application U.S. 60/791,454 filed 13 Apr. 2006, and is a continuation in part of U.S. Ser. No. 10/787,705 filed 26 Feb. 2004. Each of these applications is incorporated herein in its entirety.

### FIELD OF THE INVENTION

This invention relates to a device and method for determining parameters in fluids and, in particular, to a device for determining parameters in body fluids, such as gastrointestinal fluid parameters.

### BACKGROUND OF THE INVENTION

Many critically ill patients cannot tolerate nasogastric tube feeding, developing manifestations of intolerance, which may include nausea, vomiting, abdominal distension, aspiration and aspiration pneumonia. Indeed, it is estimated that of the 1.2 million or more patients who receive enteral tube feeding in the United States each year, up to 50% will develop aspiration pneumonia (Disario, J. A. (2002) J. Parenter. Enter. Nutr., 26, 575-579; Delegge M. H. (2002) J. Parenter. Enter. Nutr., 26, 519-525). Many of those affected patients will die as a result of their condition, or consequential complications. Prevention of such nasogastric tube feeding problems and early diagnosis may not only save thousands of lives every year, but will save millions of dollars in healthcare costs.

Since the incidence of aspiration is closely correlated with the incidence of over-feeding through enteral tubes, knowledge of the quantity of gastric fluid in the stomach (“gastric residual volume”) of a patient at any period of time is of critical importance. Gastric residual volume is a dynamic balance between fluid input (saliva, stomach secretions and feeding formula) and fluid output (gastric emptying) and, therefore, it can vary dramatically between patients fed under the same conditions. Hence, in order to effectively and safely conduct nasogastric feeding, an estimation of gastric residual volume for each patient is often used to evaluate feeding tolerance and gastric emptying. A high gastric residual volume raises concerns about intolerance to gastric feeding and the potential risk for regurgitation and aspiration pneumonia. Values of gastric residual volumes cited for patients receiving nasogastric feeding typically range from 75 to 500 ml. However, there is a great deal of controversy over the accuracy of these measurements, as discussed below.

The conventional method of aspirating the gastric contents into a syringe to give an estimate of the contents of the stomach is widely accepted to be both inaccurate and unreliable, because it is impossible to be sure that the entire stomach contents have been collected and that there is consistency from one measurement to the next. Therefore, the true gastric residual volume at any given time could be significantly different to the estimated volume. In addition, although gastric residual volumes obtained by aspiration from a nasogastric feeding tube (Asp GRVs) are widely used to evaluate tolerance to enteral feedings and gastric emptying, several reports have now shown that Asp GRVs by themselves are, in fact, poorly correlated with gastric emptying, incidence of regurgitation and the risk of pulmonary aspiration. In this regard, the conventional practice of calculating gastric residual volume typically does not take into account the fact that fluids accumulating in the stomach of the patient during nasogastric tube feeding often include not only the tube feeding formula itself, but also swallowed saliva and gastric secretions. Hence, gastric residual volumes alone cannot distinguish the additional volume of endogenous secretions in a patient who may be effectively emptying the volume of exogenous feeding.

Accordingly, the prior art use of aspirated gastric residual volumes as a monitor for gastric emptying is currently limited by poor accuracy and reproducibility due to the difficulty in aspirating the complete volume of gastric contents; and an inability to distinguish the retained volume of enteral feeding formula from any endogenous fluids that may be present. In fact, the sensitivity of conventional measurements of Asp GRVs for predicting or detecting pulmonary aspiration is only in the range of 1.9 to 8.1%.

Brix value is a measure of the soluble solid content of the fluid: it is a constant for a pure substance under standard conditions of temperature and pressure; and where there is more than one solute, the total Brix value approximates to the sum of the Brix values of the individual components. In other words, the molar fractions of the components in a mixed sample closely correlate with the Brix value contributions of each component to the total Brix value.

Brix values have been used in a number of clinical settings to determine the concentration of mixed substances such as drugs, food, fruit juices, and parenteral nutrition solutions. In comparison, there is little known about Brix values in relation to enteral nutrition solutions or its correlation to gastric emptying, which as discussed above, is extremely important in nasogastric feeding. Our co-pending US application U.S. Ser. No. 10/787,705, addresses some of these issues (as described below).

A method for accurately and reliably measuring the gastric residual volume in patients receiving nasogastric feeding, for example, is described in our co-pending US patent application U.S. Ser. No. 10/787,705. In this method, gastric residual volume is determined by a series of calculations that rely on measurements of the Brix value of the gastric fluid.

The amount of soluble solids in a fluid (e.g. Brix value) can be determined using a number of systems. For example, because the amount of soluble solids in a fluid affects its specific gravity, refractometry can be used to calculate the solids content; alternatively photodynamics (e.g. using an infra-red sensor) can be used to detect soluble components.

A handheld refractometer particularly suited for use in daylight is described in U.S. Ser. No. 10/693,904. This device comprises a light emitting diode (LED), a prism, a photoelectric sensor and a light filtering means to reduce the detection by the refractometer of external light. In addition, the refractometer includes a surface coating of a non-adhesive, wear-resistant coating to allow repeated use with corrosive or abrasive materials. The device is only suitable for measuring the refractive index of a sample and displaying the concentration or density of sugar. U.S. Ser. No. 09/842,463 describes a handheld refractometer that is intended to increase the signal-to-noise ratio detected by a scanned array of photoelectric cells. The device uses an LED light source to produce an incident light beam and a lens and mirror arrangement to deflect reflected light onto the scanned array. A reading of refractive index of the sample is obtained.

There is however no effective medical device, either based on refractometry or photodynamics, which is capable of effectively measuring a soluble solid content in a fluid, in particular, a Brix value, so as to accurately determine fluid parameters of importance in medicine, for example, gastrointestinal parameters.

Accordingly, there is a need for a medical device, system and/or method for accurately and reliably measuring gastrointestinal parameters, for example, using the method of U.S. Pat. No. 10/787,705, which may help physicians and caregivers to safely control nasogastric feeding, and which may alleviate the fear in patients that can be caused by the trauma of nasogastric feeding.

### SUMMARY OF THE INVENTION

It is an aim of the invention to provide a medical system or device for measuring at least one property of a fluid, such as the refractive index or Brix value. It is a further aim to provide methods, equations and reference databases for calculating gastrointestinal parameters such as the concentration of a dietary formula, the gastric residual volume, the amount of dietary formula retained in the stomach, the volume of dietary formula remaining in stomach, and the gastric juice volume in the stomach.

In a first aspect of the invention there is provided a test strip for use in conjunction with a medical device, wherein the medical device comprises a sample interface for measuring a property of a fluid sample, the test strip comprising: a support member having a top surface and a bottom surface and an aperture suitable for receiving the fluid sample; and wherein the test strip is configured to engage with the medical device such that the aperture of the test strip aligns with a sample interface of the medical device, thereby allowing the property of the fluid sample to be measured.

In some embodiments the test strip and/or the support member are elongate, having a longitudinal axis and a lateral axis. The longitudinal axis of the test strip or support member has a distal end and a proximal end, and at least a portion of the test trip, preferably the proximal end of the test strip is adapted to slot into a docking channel in the medical device.

Conveniently, the test strip of the invention may further comprise a system for removing fluid from the surface of the sample interface of the medical device when the test strip is removed or withdrawn from the medical device. For example, the bottom surface of the support member may be provided with a wiping system distal to the aperture, for wiping fluid from the surface of the sample interface of the medical device when the test strip is removed or withdrawn from the medical device. Advantageously, the wiping system comprises at least one resilient pad, which is compressed or deformed against the opposing surface of the medical device when the test strip is correctly engaged with the medical device, such that on withdrawing the test strip from the medical device, the resilient pad is drawn across the surface of the sample interface, to wipe any residual fluid from the surface of the sample interface. In some embodiments, the at least one resilient pad is also absorbent.

Thus, in another aspect there is provided a test strip for use in conjunction with a medical device, wherein the medical device comprises a sample interface for measuring a property of a fluid sample, the test strip comprising: a support member having a distal end and a proximal end, a top surface and a bottom surface, and being provided with an aperture suitable for receiving a fluid sample extending from the top surface to the bottom surface, and a wiping system on the bottom surface of the support member for removing fluid from the surface of the sample interface of the medical device when the test strip is withdrawn from the medical device; and wherein the test strip is configured to slidingly engage with the medical device such that the aperture of the test strip aligns with a sample interface of the medical device thereby allowing the property of the fluid sample to be measured.

In one embodiment, the test strip further comprises a sleeve adapted to enclose (for example, surround) at least a portion of the support member. The sleeve comprises at least a lower section having upwardly extending lateral flanges which engage with the support member and a window at least as large as the aperture. In such embodiments, the test strip is configured to engage with the medical device such that the window in the lower section of the sleeve aligns with the sample interface of the medical device and the aperture, such that there is a direct optical path from the sample interface through the window to the aperture.

Advantageously, the support member is slidable within the sleeve. In such embodiments, the lateral flanges of the lower section of the sleeve conveniently serve as guide members (or rails) to guide the support member through the sleeve in a substantially longitudinal direction. Most advantageously, the support member is slidable (along its longitudinal axis) within the sleeve from a first position in which the aperture is aligned With the window in the lower section of the sleeve, to a second position in which the aperture is not aligned with the window and is enclosed by the lower section of the sleeve. Thus, in the second position the aperture is not vertically aligned with the window in the lower section of the sleeve and a property of the fluid cannot be read by the medical device.

The window in the lower section of the sleeve is typically larger than or the same size as the aperture in the bottom surface of the support member. The window may be the same shape or a different shape to the aperture. For example, when the aperture is circular, the opening may be a circle, a square, or a rectangle.

Beneficially, the sleeve further comprises an upper section having an window. The upper section is engageable with the lower section of the sleeve (for example, using a snap-fit mechanism or adhesive), such that the window in the upper section aligns with the window in the lower section. In this way, the aperture in the support member can be brought into alignment with the windows in the lower and upper sections of the sleeve at the same time. Advantageously, the support member is slidable within the sleeve from a first position in which the aperture is aligned with the windows in the lower and upper sections of the sleeve, to a second position in which the aperture is not aligned with the windows and is enclosed by the lower and upper sections of the sleeve. In the second position, therefore, the aperture is preferably not vertically aligned with any part of either window in the lower or upper sections of the sleeve.

The window in the upper section of the sleeve may be larger than, the same size as, or smaller than the aperture in the top surface of the support member. Advantageously the window is the same size or smaller than the aperture. The window may be a different shape to the aperture, as described for the opening in the lower section of the sleeve. Typically, however, the opening in the upper section of the sleeve and the aperture are both circular, and conveniently, the window is substantially the same size as the aperture.

Accordingly, in a further aspect the invention provides a test strip for use in conjunction with a medical device, wherein the medical device comprises a sample interface for measuring a property of a fluid sample, the test strip comprising: an elongate support member having a distal end and a proximal end along its longitudinal axis, a top surface and a bottom surface, and being provided with an aperture suitable for receiving a fluid sample extending from the top surface to the bottom surface; and a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section; wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve; and wherein the test strip is configured to engage with the medical device such that the window in the lower section of the sleeve aligns with the sample interface of the medical device and the aperture, such that there is a direct optical path from the sample interface through the window to the aperture, thereby allowing the property of the fluid sample to be measured.

In some embodiments, the sleeve may also have a distal flange that encloses the distal end of the support member. Typically, the lower section of the sleeve is provided with an upwardly extending distal flange, which may conveniently serve to set the first position of the support member within the sleeve, i.e. it is the distal limit for the sliding of the support member within the sleeve. The second position of the support member may then be reached by sliding the support member away from the distal end portion of the sleeve.

Conveniently, in embodiments in which the test strip comprises a sleeve, the support member and sleeve are provided with a system for mutual engagement (for example, a locking mechanism), which engages when the support member is in the second position, thereby inhibiting free movement of the support member relative to the sleeve. In this way, when the aperture of the support member is housed within the sleeve any fluid within the aperture can be contained within the test strip.

Thus, in another aspect there is provided a test strip for use in conjunction with a medical device, wherein the medical device comprises a sample interface for measuring a property of a fluid sample, the test strip comprising: an elongate support member having a distal end and a proximal end along its longitudinal axis, a top surface and a bottom surface, and being provided with an aperture suitable for receiving a fluid sample extending from the top surface to the bottom surface; and a trigger which is configured to insert into a corresponding socket of the medical device and is adapted to communicate with the medical device; at least one resilient pad on the bottom surface of the support member; and a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section; wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve; wherein the test strip is configured to engage with the medical device such that the window in the lower section of the sleeve aligns with the sample interface of the medical device and the aperture, such that when the support member is in the first position there is a direct optical path from the sample interface through the window to the aperture, thereby allowing the property of the fluid sample to be measured; wherein the sliding of the support member from the first position to the second position, while the test strip is correctly engaged with the medical device, causes the at least one resilient pad to be compressed against and drawn across at least the surface of the sample interface of the medical device; and wherein the support member and sleeve are provided with a system for mutual engagement which engages when the support member is in the second position, thereby inhibiting free movement of the support member relative to the sleeve.

In certain advantageous embodiments of the test strip, a sealing system is provided on the bottom surface of the support member around the circumference of the aperture, such that, in use, when the test strip is engaged with the medical device any fluid contained in the aperture does not leak out. The sealing system may comprise a resilient material that is disposed on the bottom surface of the support member surrounding the aperture. The seal may be enhanced by a downwards force exerted by the interaction between the test strip and the medical device (e.g. a compression), when the test strip is correctly engaged with the medical device. It is particularly desirable that, in use, the seal generated between the sealing system and the sample interface (or opposing surface of the medical device) is substantially water-tight.

In further embodiments, the test strip may be provided with a handle member for ease of holding the test strip. Advantageously, the handle member is provided at or towards the distal end of the test strip. Conveniently, the handle means has a textured or contoured surface for improving grip. In additional or alternatively the handle may have a rubberised surface.

Therefore, in yet another aspect of the invention there is provided a test strip for use in conjunction with a medical device, wherein the medical device comprises a sample interface for measuring a property of a fluid sample, the test strip comprising: an elongate support member having a distal end and a proximal end along its longitudinal axis, a top surface and a bottom surface, and being provided with an aperture suitable for receiving a fluid sample extending from the top surface to the bottom surface; a handle member at the proximal end of the support member for holding the test strip; a sealing system disposed about the aperture on the bottom surface of the support member, such that when the test strip is correctly engaged with the medical device a substantially water-tight seal is formed between the aperture and the sample interface; a trigger which is configured to insert into a corresponding socket of the medical device and is adapted to communicate with the medical device; at least one resilient pad on the bottom surface of the support member; a lid for covering at least a part of the aperture in the top surface of the support member; and a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section; wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve; wherein the test strip is configured to engage with the medical device such that the window in the lower section of the sleeve aligns with the sample interface of the medical device and the aperture, such that when the support member is in the first position there is a direct optical path from the sample interface through the window to the aperture, thereby allowing the property of the fluid sample to be measured; wherein the sliding of the support member from the first position to the second position, while the test strip is correctly engaged with the medical device, causes the at least one resilient pad to be compressed against and drawn across at least the surface of the sample interface of the medical device; and wherein the support member and sleeve are provided with a system for mutual engagement which engages when the support member is in the second position, thereby inhibiting free movement of the support member relative to the sleeve.

In some embodiments of the aspects of the invention the aperture extends from the top surface to the bottom surface of the support member. The inner surface of the aperture (i.e. the surface of the support member that defines the aperture or hole in the support member) is preferably substantially non-reflective, for example, it may be black. The volume of the aperture and, therefore, the volume of the fluid sample that can be received within the aperture is typically at least 0.01 ml; 0.1 to 10.0 ml or 0.4 to 2.0 ml. In some embodiments the volume of the fluid sample may be from 0.1 to 1.0 ml or from 0.4 to 0.7 ml.

In further embodiments the test strip may comprise a lid for covering at least a part of the aperture in the top surface of the support member. Advantageously, the lid covers the greater part of the aperture.

The test strip may also be provided with means for storing and/or providing information. In such embodiments, the test strip is preferably provided with a trigger (or tip); wherein the trigger is configured to insert into a corresponding socket of the medical device and is adapted to communicate with the medical device. Advantageously, the trigger is capable of triggering a photo-interrupt in the medical device.

The trigger is preferably provided at or towards the distal end of the test strip. The trigger may be provided on the support member or on the sleeve (when present). When the sleeve is present the trigger is preferably provided on the distal flange of the sleeve.

In advantageous embodiments the test strips of the invention may be disposable.

In certain embodiments the support member of the test strip may further comprise a second aperture, and in these embodiments the test strip may be configured to engage with the medical device such that each aperture can be brought into alignment with a sample interface of the medical device.

The invention also provides a medical system for measuring a property of a fluid. The medical systems of the invention encompass a medical device capable of measuring at least one property of a fluid in conjunction with a test strip according to the invention.

Thus, by way of example, in one aspect of the invention there is provided a medical system for measuring a property of a fluid sample, comprising: a medical device capable of measuring at least one property of the fluid sample and having a sample interface for receiving a fluid sample to be measured; and a test strip; wherein the test strip comprises: a support member having a top surface and a bottom surface and an aperture suitable for receiving the fluid sample; and wherein the medical device is provided with a channel configured to receive at least a portion of the test strip, and the test strip is configured to engage with the medical device such that the aperture of the test strip aligns with a sample interface of the medical device, thereby allowing the property of the fluid sample to be measured.

In one embodiment, a medical system of the invention comprises: a refractometer capable of measuring at least the refractive index of a fluid sample and having a sample interface for receiving a sample of the fluid to be measured, and a test strip; wherein the test strip comprises: an elongate support member having a distal end and a proximal end along its longitudinal axis, a top surface and a bottom surface, and being provided with an aperture suitable for receiving a fluid sample extending from the top surface to the bottom surface; a trigger which is configured to insert into a corresponding socket of the medical device and is adapted to communicate with the medical device; at least one resilient pad on the bottom surface of the support member; and a sleeve adapted to enclose at least a portion of the support member; wherein the sleeve comprises a lower section having upwardly extending lateral flanges which are capable of engaging with and acting as guide members for the support member, and a window at least as large as the aperture; wherein the support member is slidable within the sleeve from a first position in which the aperture is aligned with the window in the lower section of the sleeve, to a second position in which the aperture is not aligned with the window and is enclosed by the lower section of the sleeve; wherein the medical device is provided with a channel to receive at least a portion of the test strip and a socket to receive the trigger of the test strip; and the test strip is configured to slidingly engage with the channel of the medical device such that the window in the lower section of the sleeve aligns with the sample interface of the medical device and the aperture, such that when the support member is in the first position there is a direct optical path from the sample interface through the window to the aperture, thereby allowing the property of the fluid sample to be measured; and wherein the sliding of the support member from the first position to the second position, while the test strip is correctly engaged with the medical device, causes the at least one resilient pad to be compressed against and drawn across at least the surface of the sample interface of the medical device.

As noted above, the test strip may comprise any of the features described herein in relation to test strips of the invention. The details of such test strips comprised in medical systems of the invention are not reproduced here. Likewise, the medical device comprised in the medical systems of the invention may include the features of any of the medical devices according to the invention (described below). The details of such medical devices are described elsewhere herein.

Beneficially, the medical device further comprises a digital display for reporting a measurement of a property of a fluid; and more preferably a keypad for manual communication with the medical device.

In preferred embodiments the medical device is a refractometer, which measures at least the refractive index of a sample of fluid. The medical device may be a hand-held refractometer.

In further embodiments, the medical system of the invention may comprise a docking station, the docking station being capable of electronic communication with the medical device for downloading and/or uploading data electronically. The docking station may be particularly advantageous when the medical device is a hand-held refractometer.

When the medical device is a refractometer, the refractometer preferably comprises means for converting a measurement of a refractive index of a fluid into a measurement of Brix value (BV) of the fluid. The conversion is typically performed electronically, preferably automatically (e.g. at the press of a button). The means of converting a refractive index of a fluid into a measurement of Brix value (BV) of the fluid is conveniently stored in the device, for example, in the form of one or more standard (calibration) curves, look-up tables, equations and the like.

The medical device or refractometer preferably further comprises means for calculating gastrointestinal parameters such as the gastric residual volume (GRV), the concentration of dietary formula, the amount of dietary formula remaining in the stomach, and/or the gastric juice volume in the stomach in a subject based on the measured Brix values (BV) of samples of gastric fluid.

In one embodiment, the medical device comprises means for calculating GRV in a subject based on the measured BVs of at least two samples of gastric fluid, at least one of which samples was obtained from the subject after the gastric contents had been diluted in situ with a known volume of a fluid; wherein the means for calculating the GRV in the subject calculates the GRV using the equation: GRV=(BV2×Vol)/(BV1−BV2); wherein BV1 is the measured pre-dilution BV of a sample of gastric fluid before dilution of gastric contents with a known volume of fluid (Vol), and BV2 is the measured post-dilution BV of a sample of gastric fluid after dilution of gastric contents with the known volume of fluid (Vol).

It will be appreciated that the medical systems or kits of the invention may comprise a plurality of test strips.

The invention also relates to an optical module for use in a refractometer, particularly for use in the methods of the invention.

Thus in one aspect of the invention there is provided an optical module for a refractometer, comprising: a prism having a sample interface for contacting a fluid sample to be measured; a light source arranged to project light through a first face of the prism onto the sample surface; and an array of photoreceptors arranged proximal to a second face of the prism to detect light reflected from the sample interface when a fluid sample is in contact with the sample interface; wherein the light source is a laser diode; and wherein the array of photoreceptors comprises one or more photodiodes.

The prism of the optical module is preferably made from a material selected from the group consisting of glass, quartz and plastic.

Conveniently, the wavelength of the laser is from 600 to 1300 nm, preferably from 650 to 850 nm and more preferably from 700 to 800 nm. Typically, the laser diode projects light onto the sample interface of the prism at an angle of incidence of 42 to 62°, preferably from 50 to 62° and more preferably from 55 to 62°.

Advantageously, the array of photoreceptors comprises two photodiodes. In such embodiments, a first photodiode may be arranged to detect refracted light and a second photodiode may be arranged to detect scattered light. Such embodiments provide the advantage that the optical module (and hence a refractometer comprising the optical module) can be used with heterogeneous fluids, such as suspensions or emulsions, rather than only with homogeneous solutions.

The invention further provides a kit comprising a plurality of test strips according to the invention, and may further comprise instructions for calculating at least one gastrointestinal parameter in a subject, for example, gastric residual volume in a patient receiving enteral tube feeding.

In a further aspect there is provided a medical device for measuring gastric residual volume (GRV) in a subject, comprising: means for receiving a sample of gastric fluid that has been aspirated from the subject; means for measuring the refractive index of the sample of gastric fluid; means for converting the refractive index measurement into a Brix value (BV) for the sample of gastric fluid; means for calculating the GRV in the subject based on the measured BVs of at least two samples of gastric fluid, at least one of which samples was obtained from the subject after the gastric contents had been diluted in situ with a known volume of a fluid.

Advantageously, the means for converting the refractive index measurement into a Brix value (BV) for the sample of gastric fluid comprises a reference database. The reference database is beneficially stored electronically and may comprise at least one predetermined look-up table, equation or standard curve.

In some embodiments the means for calculating the GRV in the subject calculates the GRV using the equation: GRV=(BV2×Vol)/(BV1−BV2); wherein BV1 is the measured pre-dilution BV of a sample of gastric fluid before dilution of gastric contents with a known volume of fluid (Vol), and BV2 is the measured post-dilution BV of a sample of gastric fluid after dilution of gastric contents with the known volume of fluid (Vol).

In yet another aspect there is provided the use of a medical device suitable for measuring the refractive index of a fluid for calculating the gastric residual volume (GRV) in a subject, said use in an in vitro method which may comprise the steps of: measuring the refractive index of a sample of gastric fluid from the subject to obtain a pre-dilution refractive index; converting the pre-dilution refractive index into a pre-dilution Brix value (BV1); measuring the refractive index of a sample of gastric fluid from the subject after the gastric contents had been diluted with a known volume of fluid (Vol) to obtain a post-dilution refractive index; converting the post-dilution refractive index into a post-dilution Brix value (BV2); and calculating the GRV by multiplying the post-dilution Brix value (BV2) by the known volume of water (Vol) and dividing the resulting product by the difference between the pre-dilution Brix value (BV1) and the post-dilution Brix value (BV2).

These and other aspects and features of the invention will be more fully appreciated when the following detailed description of the invention is read in conjunction with the accompanying drawings. For example, it will be appreciated that all medical device embodiments and/or all test strip embodiments may be comprised with the medical systems of the invention; and all optical module embodiments may be comprised within the medical device and medical systems of the invention.

All literatures and patents mentioned hereinabove, as well as the literatures cited herein, as incorporated herein by reference in their entirety.

### BRIEF DESCRIPTION OF THE DRAWINGS

FIG. 1 is a perspective view of the medical device according to the invention;

FIG. 2 is a block diagram of a system of operation of a medical device according to the invention;

FIG. 3 shows a perspective view of a medical system according to the invention comprising a medical device and a test strip (A); and a medical device, test strip and docking station (B);

FIG. 4 shows a front view of a test strip according to the invention (A); a rear view of the test strip shown in FIG. 4A (B); another embodiment of a test strip according to the invention having a trigger (C); and a different embodiment of a test strip according to the invention having a handle means (D);

FIG. 5 is an exploded perspective view of a preferred embodiment of a test strip according to the invention;

FIG. 6 is a perspective view of a preferred medical system according to the invention comprising the test strip of FIG. 5 and a hand-held refractometer;

FIG. 7 is a flow chart summarising an overall process employing a medical system and method in accordance with an embodiment of the invention; and

FIG. 8 is a schematic representation of a preferred optical module for use in a medical device according to the invention.

### DETAILED DESCRIPTION OF THE INVENTION

Prior to setting forth the invention a number of definitions are provided that will assist in the understanding of the invention.

As used herein, the term “Brix value” refers to a constant for a pure substance under standard conditions of temperature and pressure, and closely correlates with the molar fractions of the components in the test sample, in particular, a fluid. In other words, the overall Brix value of a mixed solution approximates the additive sum of the Brix values of its individual components.

As used herein, the term “a test sample”, “a sample (of a fluid)”, and “a specimen fluid” are interchangeable and refer to the (typically small) quantity of fluid that is to have at least one property (such as refractive index) measured or tested by the medical device or system of the invention to have its soluble solid content.

As used herein, the term “dietary formula” refers to the dietary formula for gastric tube feeding or any type of enteral nutrition, comprising polymeric diet formula or liquid dietary formula.

The phrase “gastric residual volume” or “GRV” refers to the volume of the gastric contents in a subject (or patient) at that point in time. By way of example: if the stomach of a subject is completely empty when 100 ml of diluted dietary formula is added, and 10 minutes later 10 ml of gastric juices and saliva has been introduced into the stomach, but 30 ml of the fluid has been removed from the stomach, then the gastric residual volume would be 80 ml.

Methods for Measuring Parameters of a Fluid

The present invention relates to a system and method for determining gastrointestinal parameters, in particular, the soluble solid content of dietary formula, the gastric residual volume, and the nutrition intake related to nasogastric feeding to the patients.

Concentration of Dietary Formula

In one method described herein the concentration of dietary formula in the stomach of a subject is measured. While the concentration of dietary formula infused into the stomach may be known, once a period of time has elapsed following the introduction of the dietary formula, it is likely that the composition and concentration of dietary formula in the stomach (i.e. gastric contents) will be different to the fluid infused.

Infusing of dietary formula into the stomach of a subject (or patient) may be accomplished by nasogastric feeding, enteral nutrition feed, or by any known is tube feeding technique. The methods of the invention can be applied with any dietary formula known to the person skilled in the art. For example, the dietary formula may be a dietary formula for gastric tube feeding or any type of enteral nutrition. In another embodiment, the dietary formula may be a liquid dietary formula or a polymeric dietary formula. In addition, the methods of the invention are suitable for use with any concentration of dietary formula; that is, dietary formula with a concentration of 0 to 100% can be measured/monitored by the medical devices of this invention. For the avoidance of doubt, full-strength polymeric dietary formula would be considered to be a 100% concentration of dietary formula.

In the first instance, the medical device or system of the invention is used to measure the soluble solid content of the infused dietary formula; i.e. the refractive index of a sample of fluid that has previously been extracted (e.g. by aspiration) from the stomach of a subject (or patient). In a preferred embodiment, the value of refractive index is converted into a Brix value (BV), preferably directly by the medical device or system used in accordance with the invention. The skilled person will appreciate that any suitable means of converting a measured refractive index into a BV can be used. For example, an electronic or manual look-up table (conversion chart), mathematical equation or graph can be used which correlates a refractive index for a fluid containing dietary formula from a refractive index measurement to a BV. Hence, where reference is made herein to a measured or calculated BV, the BV is typically obtained by directly measuring the refractive index of the sample and then converting the refractive index measurement to a corresponding BV as indicated above.

In a preferred method, a slope value (i.e. gradient or correlation) is first derived from BVs of a known, serially diluted dietary formula over a predetermined concentration range (e.g. from 0 to 100%). In this way, a standard set of data (e.g. a standard curve) for the relationship between the concentration of dietary formula and its BV can be obtained for the particular dietary formula. The measured or calculated BV for a test fluid (e.g. a sample of gastric content) can then be compared to the standard set of data (or curve) to determine the concentration of dietary formula in the gastric content sample.

By way of example, the calculated BV of a dietary formula suitable for enteral infusion is plotted against serial dilutions of that dietary formula or polymeric dietary formula (e.g. expressed as concentration percentages of the full-strength dietary formula). Typically, a plurality of different concentrations of dietary formula and corresponding BV measurements are used to create the standard data set for each type of dietary formula. Thus, BVs may be determined for 5 dietary formula concentrations, e.g. 0, 25, 50, 75 and 100%; for 6 concentrations, e.g. 0, 20, 40, 60, 80 and 100%; or for more than 6 concentrations, e.g. for 6 to 10 different concentrations. The slope value (gradient) for each standard curve for each dietary formula is calculated according to known regression analysis methods.

Preferably, the serial dilutions of dietary formula for preparation of standard data sets are carried out by diluting the dietary formula into a solution that has a very low refractive index. In some embodiments, the dietary formula may be diluted with water, distilled water, gastric juice, saliva, saline solution (weak sodium chloride solution) or dextrose solutions. However, other suitable diluents may be used. In a preferred method the diluent is distilled water. In a preferred method the slope value is 0.24.

It has been found that the difference in the refractive index between dietary formula that has been diluted with distilled water, saline or gastric juice has a negligible affect on the resultant refractive index and calculated concentration of the dietary formula. Advantageously, therefore, the concentration of dietary formula in a gastric content sample can be accurately measured by reference to a standard dilution series that was prepared by dilution of dietary formula in, e.g. is distilled water.

Accordingly, an in vitro method of determining the concentration of dietary formula in a subject is provided, the method comprising: optionally obtaining an infused dietary formula from a subject e.g. by aspiration (the dietary formula may have previously be obtained from the subject); measuring a BV of the infused dietary formula; determining a slope value derived from BVs of serially diluted dietary formula over a determined concentration range; and dividing the BV of the infused dietary formula by the slope value, In other words, the percent concentration of dietary formula may be expressed as: % concentration of dietary formula=(BV of infused dietary formula I slope value)

The Brix values of polymeric dietary formula have a linear additive relationship with the dietary formula concentration (R2=0.99). With such a high degree of correlation, the measured Brix value may be correlated to the percent concentration of the formula (e.g. % full-strength polymeric dietary formula) at any dilution.

Gastric Residual Volume (GRV)

In another method according to the invention, the gastric residual volume (GRV) of a subject may be determined. Thus, the soluble solid content of a first gastric content sample is measured (e.g. the refractive index) and then the BV is determined, for example, on the basis of the measured refractive index. For convenience the BV of the first gastric content sample (which may be considered a pre-dilution BV) is termed BV1. Preferably, the BV is measured using a medical device according to the invention.

As previously mentioned, the gastric content sample may, for example, contain dietary formula, bodily secretions (e.g., gastric juices and/or saliva), and mixtures thereof, without adversely affecting the method of the invention. The sample may be an in vitro gastric content sample previously obtained from a subject, or the method may involve obtaining (e.g. by aspiration) a sample from the stomach of a subject (or patient).

A known volume of liquid (e.g. water) is then either: (i) added to the pre-dilution gastric content sample and reintroduced into the stomach of the subject to form a diluted gastric content; or (ii) added directly into the stomach of the subject (for example, where the pre-dilution sample had been stored in vitro before the measurement of BV was made) to form a diluted gastric content. Any suitable amount of liquid may be added to the sample or directly to the subject depending on parameters that will be appreciated by the person skilled in the art, such as the size of the sample, the type of dietary formula, the condition of the subject, etc. The volume of liquid used to dilute the gastric contents may, for example, range between 10 ml and 500 ml. The diluted pre-dilution gastric content sample or water is infused into the stomach using known infusion methods.

In the next step a second “post-dilution” gastric content sample is obtained from the subject, e.g. by aspiration; or alternatively a second “post-dilution” gastric content sample has previously been obtained from the subject. The BV of the post-dilution sample (BV2) is then measured or calculated in a similar way to that for the pre-dilution sample.

Finally, the GRV may be expressed as: GRV equals BV2 multiplied by volume of added liquid (Vol), and divided by the difference between BV1 and BV2.

By way of example: the gastric residual volume (i.e. the volume of stomach contents at the start of the method) may be set as Vol.1; the % full-strength dietary formula as % Conc.1; and the BV of gastric contents as BV1. After the first step in the method, a known volume of liquid (e.g. 30 ml water) is added to the previously obtained gastric content sample, which is then infused back into the stomach of the subject, for example, using a nasogastric tube. The new gastric volume can then be considered to be Vol. 2; the % full-strength dietary formula as % Conc.2; and the BV of the diluted gastric contents as BV2. Before the second gastric content sample is obtained for measurement of BV2 it is advantageous to mix the diluted stomach contents, e.g. by repeated aspiration, to evenly dilute the gastric contents. Thereafter, a second “post-dilution” gastric content sample is obtained for measurement of BV2. The gastric residual volume (Vol.1) may then be calculated according to the equation:
Vol.1=(BV2×30 ml)/(BV1−BV2).
Accordingly, the invention provides an in vitro method for determining gastric residual volume in a subject, the method comprising: (a) measuring the BV of a gastric content sample to obtain a pre-dilution BV (BV1); (b) adding a known volume of liquid to the gastric content sample and returning the diluted sample to the stomach of the subject to form a post-dilution gastric content; (c) obtaining a post-dilution sample; (d) measuring the BV of the post-dilution sample to obtain a post-dilution BV (BV2); and (e) multiplying the BV of the post-dilution gastric content sample by the known volume of liquid and dividing the resulting product by the difference between the pre-dilution BV and the post-dilution BV.
Gastric Juice Volume

A method of determining gastric juice volume in the stomach is also disclosed by: measuring the dietary formula volume remaining in the stomach of a subject, calculating or measuring the GRV, and determining the difference between the GRV and the dietary formula remaining in the stomach. The volume of dietary formula remaining in the stomach can be obtained by determining the concentration of dietary formula in the stomach and the GRV, as disclosed above. Hence, the described methods can also be used to determine whether gastric contents are comprised predominately of dietary formula or digestive secretions in patients receiving polymeric dietary feeding, because: (i) endogenous secretions (such as saliva and gastric juice) do not mask the BV due to dietary formula; and (ii) the dilution of dietary formula in the stomach does not adversely affect the calculation of dietary formula based on standard dilution series of dietary formula.

In addition, the dietary ranges of nutrients (such as carbohydrate, protein and fat) may be monitored using the systems and methods of the invention. In this regard, the BV of a solution is a linear additive function of the concentration of nutrients present in solution and BVs correlate with the concentrations of dietary formula independent of pH, temperature, and the type of diluent solution (as mentioned above). Thus, BV can be used clinically and in research to monitor dietary formula concentrations and, therefore, it can be used in clinical practice to evaluate dietary formula during storage, preparation and administration.

The BV measurement of a gastric content sample can be used to monitor both GRV and food content in patients receiving enteral nutrition, e.g. by nasogastric feeding.

Gastric Content Emptying and Feeding Tolerance

Furthermore, the BV measurement of gastric juice can be used to monitor gastric emptying in patients receiving nasogastric feeding, thereby providing additional information beyond the simple measurement of an aspirated gastric residual volume. Thus, a method of monitoring gastric content emptying and tolerance in patients receiving dietary formula is also disclosed.

The BV ratio can be used as an alternative measure of the amount of dietary formula retained in the stomach, to monitor dietary formula processing by a subject receiving enteral tube feeding. The BV ratio is calculated as: (post-dilution Brix value)/(pre-dilution Brix value). In this way, the rate at which the dietary formula is naturally removed from the stomach of a subject receiving enteral tube feeding can be monitored; and the tolerance of the subject to enteral tube feeding can thereby be assessed.

In the alternative, feeding tolerance may be monitored by measuring the volume of dietary formula remaining in the stomach using a method according to the invention, as described below.

As previously described, the pre-dilution Brix value may be obtained from measuring the Brix value of a sample aspirated from the gastric contents of the stomach of a patient infused with dietary formula, before the stomach contents are diluted with a known volume of water. After a known volume of water has been added to the gastric contents by infusion into the patient, preferably together with the previously obtained pre-dilution sample, the post-dilution BV may be determined by obtaining a second sample of gastric contents (e.g. by aspiration) and measuring the new BV.

The specific component of gastric residual volume that is volume comprised of dietary formula may be determined using the equation: Volume of dietary formula remaining in stomach=[calculated GRV×(pre-dilution BV/slope value)]/100. It will be appreciated that the value of (pre-dilution BV/slope value) is the % concentration of dietary formula in the gastric contents. In one embodiment the slope value is 0.24.

In summary, since BV measurements for various dilutions of the polymeric dietary formula have minimal variability in vitro, the disclosed methods permit bedside measurements with a high degree of reproducibility.

Medical Devices for Measuring Parameters of a Fluid

The theoretical basis of using BVs in determining Gastrintestinal Parameters has been published in our co-pending US patent application U.S. Ser. No. 10/787,705; and in Chang W.-K. et al. (2003) Monitoring bolus nasogastric tube feeding by the Brix value determination and residual volume measurement of gastric contents, J. Parenter Enter Nutr., 28(2), 105-112.

In this work a refractometer was used to measure the refractive index of gastric contents and to then determine the BV of the sample of fluid. A typical handheld refractometer, such as that previously used is Mode Model N.O.W. 507-1; Nippon Optical Works, Tokyo, Japan.

Refractometry

Refractometry is a useful technique because of its minimal expense and ease of utilisation. A preferred medical device for use in accordance with the invention is a refractometer.

A refractometer can be used to measure the refractive index of a material, such as a sample fluid. The refractive index of a substance is a measure for how much the speed of light (or other wave) is reduced inside the substance, in comparison to its speed in air or a vacuum. When light waves cross the interface between two materials of different refractive index the light waves change direction; also the light beam reflects partially at the interface of the two materials.

Brix Value (BV) is a measurement of total soluble solids in a solution. The BV of a solution (or fluid) can be obtained by converting the refractive index of the solution, which can be measured using refractometry by a refractor. This value is a constant for a pure substance under standard conditions of temperature and pressure. Moreover, BV closely correlates with the molar fractions of the individual components in the solution. In other words, the overall BV of a mixed solution approximates to the additive sum of the BVs of its individual components. BVs have been used in a number of clinical settings to determine the concentration of mixed substances such as drugs, food, fruit juices, and parenteral nutrition solutions. However, there is not much known about the relation of BV to enteral nutrition solutions or its correlation to gastric emptying. Our co-pending application U.S. Ser. No. 10/787,705 relates to the determination of BV for dietary formulas, the parameters that affect BV measurements of gastric contents and the correlation between Brix values and gastric content components. Thus, refractometry has been shown to be a useful tool in clinical settings and, in particular, for measuring gastrointestinal parameters. However, the present commercial refractometers have limitations for medical use, in particular, for determining gastrointestinal parameters.

In this regard, most existing commercial refractometers operate on the principle of Snell's Law of Refraction, which compares the difference in the refractive indexes of two materials, for example, with refractive indexes n₁ (which may be the refractive index of a prism in the refractometer) and n₂ (which refers to the refractive index of the material to be tested, such as sucrose solution, water, dietary formula and so on), as a light beam strikes the interface between the two materials. Typically, the sample to be tested is placed directly on the sample interface of the prism of the instrument; a light beam is directed through the prism onto the sample interface; and the sample refracts the light beam such that the angle of refraction (i.e. the angle of the light that travels through the sample) is different to the angle of incidence (i.e. the angle at which the light beam strikes the interface). Any net deflection of the light beam can be used to determine the refractive index of the sampler.

Fresnel's equation, however, relates to the second affect that is exhibited at the interface between two materials having different refractive indexes when a light wave strikes the interface; i.e. the proportion of light that is reflected at the interface between the materials.

The invention provides an improved optical module based on Fresnel's equation, for use in a refractometer.

In addition, the invention provides a test strip for use in conjunction with a medical device, such as a refractometer, for measuring a property of a fluid sample. Also provided are medical devices that operate according to the methods of the invention, and medical systems that comprise medical devices and/or test strips according to the invention. Embodiments of the invention will now be further described with reference to the accompanying figures.

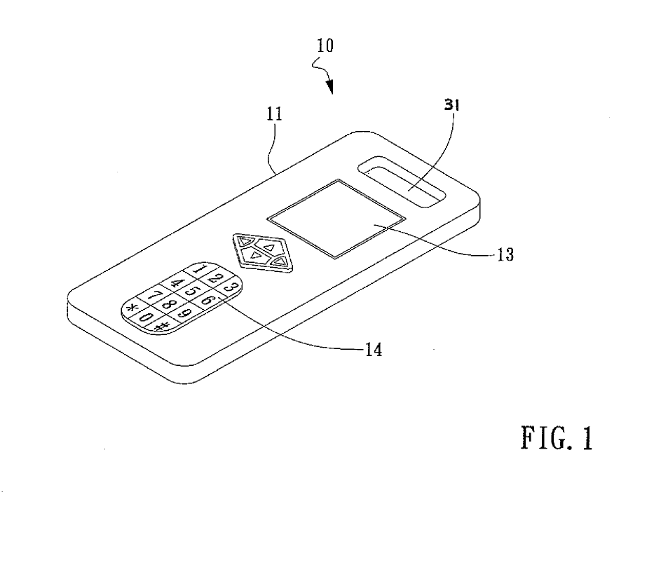

FIG. 1 is a perspective view of a medical device 10 according to a preferred embodiment of the present invention. The medical device 10 (which may be a refractometer and in some embodiments a hand-held refractometer), includes a housing 11, a detecting area 31, a display 13, (preferably a digital display) and a keypad 14. The medical device 10 of this invention is manufactured to detect a soluble solid content in a fluid sample placed above the detecting area 31. In this embodiment, the sample may be placed directly in the detecting area 31 and once the required measurement(s) of one or more sample fluid properties have been taken the sample must be removed from the detecting area 31. It is necessary to clean the remnants of the fluid sample from the detecting area 31 before a new sample is placed in the detecting area 31. The sample may include a dietary formula, gastric juice, liquid for injection or transfusion or other similar fluid. Preferably the fluid is a gastric content sample or dietary formula. The display 13 could be a liquid crystal display (LCD) or any other suitable type, which is disposed on the exterior of the housing 11, and coupled to the detecting area 31 to display a measurement that has been determined; e.g. of refractive index, advantageously of BV, or in some embodiments properties of body fluids, such as gastrointestinal parameters. The keypad 14 is also connected to the display 13, and is used, for example, to respond to instructions appearing on the screen 13, or to instruct the device to carry out certain measurements or programs, as desired. It is envisaged that certain of the above-mentioned elements of the medical device 10 could be provided separately from the medical device or replaced by any compatible elements. By way of example, the keypad 14 and the display 13, could be replaced by a personal digital assistant (PDA) or a computer connectable to the medical device.

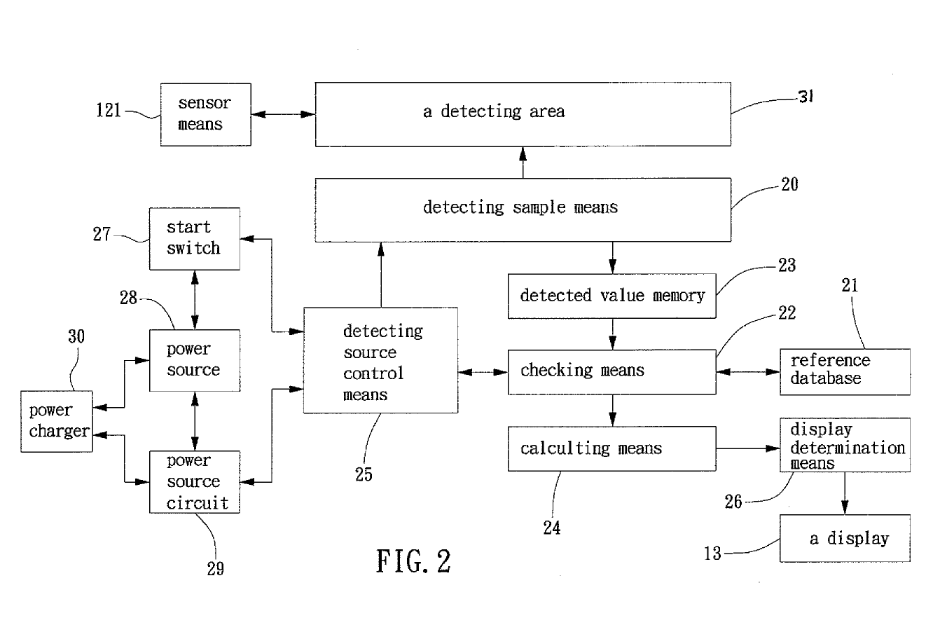

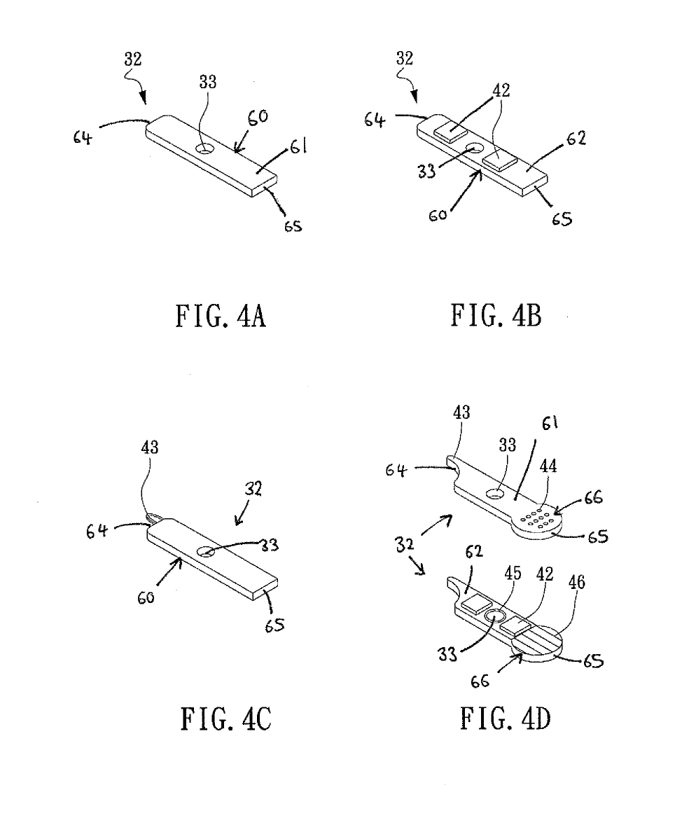

FIG. 2 is a block diagram of the mode of operation of the medical device according to a preferred embodiment of the present invention. A detecting sample means 20 connected to a detecting area 31, could be arranged for refractometry or photodynamic measurements. In one embodiment, the detecting area 31 is further coupled to a sensor means 121, which detects whether a sample is present to be measured. The sensor means 121 will be described in more detail in FIG. 4C and 4D. The detecting sample means 20 is linked to a detected value memory 23 that interacts with a checking means 22. The checking means 22 compares the detected value measured and calculated by the medical device to any values in the reference database 21, to determine whether the value measured is qualified or not. Amongst other things, the reference database 21 may store the means of converting a signal of refractive index into a BV (if required), equations necessary to calculate gastrointestinal parameters, or alternative means for calculating e.g. the concentration of dietary formula, the gastric residual volume (GRV), the amount of dietary formula retained in the stomach, the volume of dietary formula remaining in stomach, and the gastric juice volume in the stomach. Preferred reference databases include look-up tables, conversion charts and equations, as previously described. Once the checking means 22, has verified that the detected value is qualified, a required property or parameter of the sample is determined by a calculating means 24. If however the detected value is not qualified the checking means 22 sends a warning message to a detecting source control means 25; and the reference database 21 may re-test the sample. After the required parameter has been obtained by the calculating means 24, the result of the measurement (optionally along with a confirmation that the measurement was correctly made) is prepared by a display determination means 26, and transmitted to and shown on the display 13. Typically, the medical device also has a start switch 27, which is connected to a power source 28 for switching the medical device on and off. The power source 28 is connected to a power source circuit 29, which is electrically coupled to the detecting source control means 25, so that the detecting sample means 20 can be turned off and on as required. The power source 28 may be any suitable means of providing power. In a preferred embodiment, the power source 28 is a rechargeable battery system that is capable of being recharged by a power charger 30. The power charger is typically powered by household electric current (for example, 110/220 volts). In another embodiment, the power source 28 is in a disposable type, such as a disposable battery; in such a circumstances, the power charger 30 may not be necessary. In an alternative mode, the power source 28 and power charger 30 may be arranged to operate synergistically, as indicated in FIG. 2, such that the power source circuit 29 can receive power from a battery or directly from mains (household) electric power. Thus, in one embodiment the medical device of the invention can operate from an external power source (such as an external electricity supply). Preferably, in another embodiment, the power source of the medical device is designed to be able to smoothly switch between the battery system and the external power source.

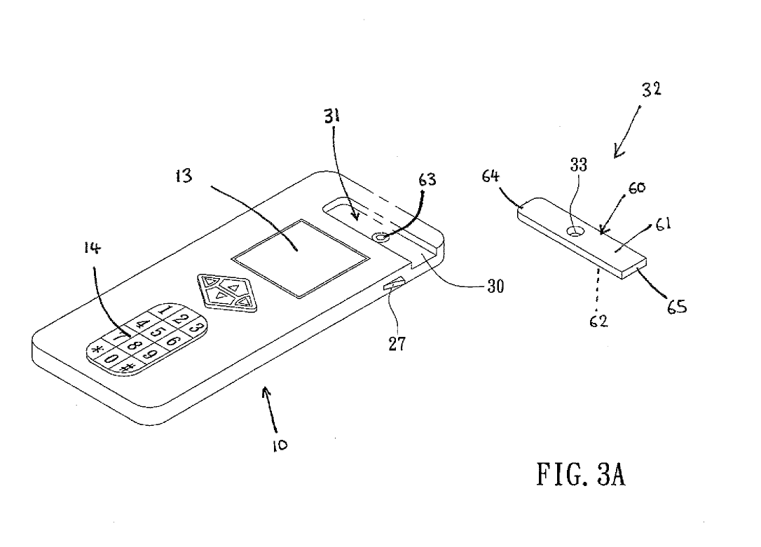

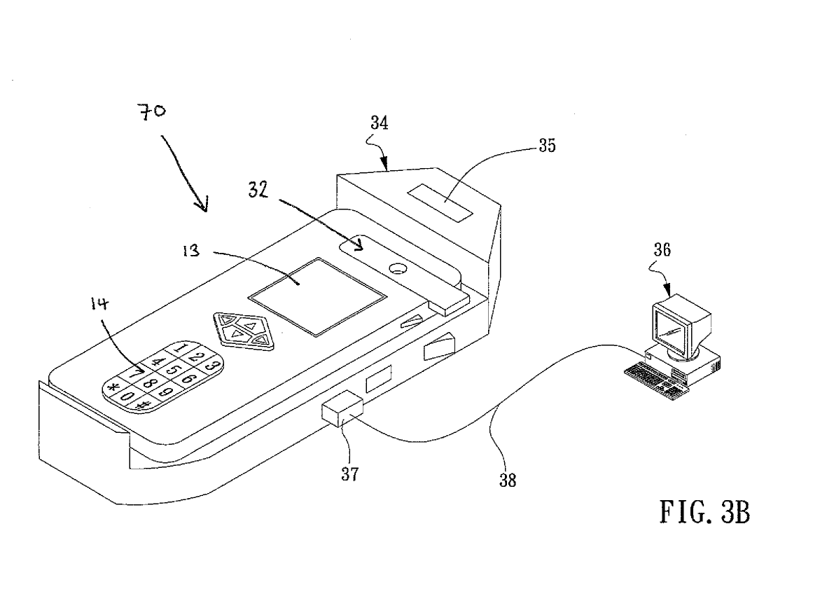

FIG. 3 shows a perspective view of a medical system according to an embodiment of the invention. In (A) the detecting area 31 of the medical device 10 of FIG. 1 is shown in more detail, along with a test strip 32 according to an embodiment of the invention.

The test strip 32 is in the form of an elongate support member 60 having a top surface 61 and a bottom surface (62, not shown); a distal end 64 and a proximal end 65. The support member 60 is provided with an aperture 33, extending from the top surface 61 to the bottom surface 62 of the support member. In the embodiment depicted the aperture is circular, however, it will be appreciated that the aperture may be of any shape (e.g. substantially square, oval, rectangular or hexagonal), provided it is suitable for receiving a fluid sample to have a parameter measured by the medical device 10. Typically, the volume of the aperture is less than 10.0 ml and more than 0.01 ml. Advantageously, the aperture is between 0.1 and 10.0 ml, and more advantageously between 0.4 and 2.0 ml. In some embodiments the aperture may not extend through the bottom surface (not shown) of the support member 60, provided that a fluid sample in the aperture can still be detected by the medical device 10. In other words, it is necessary for there to be a direct optical path between the detecting area 31 of the device 10 and the aperture 33. Thus, for example, the bottom of the aperture may have a transparent covering, such as a layer of glass, plastic or quartz. This prevents fluid from leaking from the aperture and means that a sample of fluid can be inserted in the aperture even before the test strip 32 is placed onto the detecting area 31 of the medical device 10. In an alternative embodiment (not shown), the top of the aperture may not be open. For example, the aperture may extend from the bottom surface of the support member 60 only part-way through the depth of the support member 60. This provides the advantage that, in use, light cannot penetrate through the aperture and the sample of fluid into the detecting area 31. However, in this case the sample of fluid is preferably prevented from leaking from the aperture 33 by surface tension. In such circumstances the aperture may have a smaller volume. In a preferred embodiment, the aperture extends from the top surface 60 to the bottom surface of the support member 60.

The detecting area 31 of the medical device 10 has an upwardly facing sample receiving area, which may be in the form of a sample interface 63. The sample interface 63 is advantageously a flat surface: typically the surface of a prism that forms a part of the optical module of the medical device 10. A sample of fluid should be placed into contact with the surface of the sample interface 63 in order for a parameter of the fluid to be measured.

It will be appreciated that the detecting area 31 may be designed in any shape or size, provided it is suitable for taking a measurement of a parameter of a sample of a fluid presented to the medical device on a test strip 32 in accordance with the invention. Thus, the detecting area 31 and the test strip 32 are configured to engage in such a way that (when correctly engaged) the aperture 33 of the test strip aligns with the sample interface 63 of the medical device 10. Generally, the sample interface 63 is upwardly facing and thus, the aperture 33 and the sample interface 63 may be considered to be vertically aligned. The detecting area 31 is designed with a means for engaging a test strip 32 in the form of a channel 30, which in the embodiment depicted is an open groove or channel having an opening on a lateral side of the medical device 10 to enable the insertion of the test strip 32. Conveniently, the channel 30 may be referred to as a “strip holder”, because that is its function. In alternative embodiments the strip holder may comprise an enclosed channel (rather than an open groove) or a slot, and it may be open on a lateral side, on the top, bottom or at an end of the medical device 10, to allow insertion of the test strip 32. The type of strip holder determines the manner in which the test strip must be inserted. For example, in the embodiment depicted in FIG. 3A, the test strip may be pushed downwards into the channel 30, or more typically, may be slid along the channel 30 until the distal end 64 abuts the end of the channel 30. Thus, the test strip is advantageously configured to be a slide-fit in the channel 30 of a medical device 10. In some embodiments, the channel is at least partially enclosed (i.e. covered at the top), which has the advantage that the amount of external light entering the detecting area 31 is reduced.

In use, at least a portion of the test strip 32 is placed in the detecting area 31 such that the aperture 33 of the test strip 32 is vertically aligned with the sample interface 63 of the medical device 10. A sample of a fluid to be tested is then placed in the aperture 33 of the test strip 32, and the fluid contacts the upwards facing surface of the sample interface 63. When the medical device 10 is ready to measure a parameter of a sample of test fluid in the aperture 33, an internal light (advantageously in the form of a laser) is directed at the underneath of the detecting area 31 and the light will strike the interface between the sample and the detecting area 31 at the sample interface 63. As noted previously, the sample interface 63 is typically a surface of a prism that forms a part of the optical module of the medical device 10. A measured parameter of the fluid sample may then be displayed on the display 13 of the medical device 10.

In FIG. 3B a medical system 70 according to one embodiment of the invention is shown. The medical system 70 may, for example, be used for monitoring the nutrition intake condition of a subject (for example, a patient). In a basic form, a medical system 70 of the invention may comprise just a medical device 10 and one or more test strips 32, as previously described. However, in one embodiment the medical system 70 further comprises a docking station 34 (or desk-top unit) having means for electronic communication with the medical device 10, for example, by way of a connection port (not shown). Conveniently, the docking station 34 may be used to recharge the batteries in the medical device 10, when rechargeable batteries are used. In this case, the docking station 34 is capable of delivering full-rated electrical current (e.g. mains) when it is necessary or desirable to recharge the batteries in the medical device 10. The docking station 34 may further comprise a recognition system, including but not limited to a barcode reader, or radio frequency identification (RFID; both not shown), for recognising or reading a code, such as to verify the identity of the user of the device or corresponding to information stored on the test strip 32. In this way, private information of the user, or any confidential or security functions can be maintained. The docking station may also comprise a paper exit (or port) 35 for printing out a hard copy of the results recorded by the medical device 10. In alternative embodiments, the docking station 34 may have its own display means and/or keyboard for communication and recording purposes. Furthermore, the docking station 34 may be provided with electronic storage and data processing systems to enable the processing of data that may be transferred from the medical device 10. For example, where the medical device 10 is a basic refractometer, the docking station may be used to convert a reading of refractive index into a BV measurement, or a reading of GRV and so on.

The medical system 70 may optionally further comprise a computer 36 that is connectable to the medical device 10 and/or the docking station 34. The connection system can be any suitable means, for example, wireless or an electrical cable 38 and computer connector 37. In this way the processing capacity of the medical system 70 can be greatly enhanced to process and store large amounts of data rapidly, and to receive and optionally combine data from separate sources either simultaneously or sequentially. In addition, by using different detecting functions of the medical device 10 and multiple connecting ports on the docking station 34 (not shown), or the computer 36; the medical system 70 can be connected to other compatible machines to monitor the condition of a subject or patient for any suitable purpose. The computer 36 could, in alternative embodiments, be substituted for a PDA.

FIGS. 4A and 4B show front and rear views, respectively, of a test strip 32 as previously described in relation to FIG. 3A. Briefly, the test strip 32 comprises an elongate support member 60 having a distal end 64 and a proximal end 65, and an aperture 33 suitable for containing a sample of a fluid, which extends from the top surface 61 to the bottom surface 62 of the support member 60. The support member 60 should preferably have sufficient rigidity such that it does not bend when supported only at either the distal or proximal end 64, 65, respectively. In addition, the support member 60 should have sufficient rigidity to enable its insertion into the channel 30 (not shown) of a medical device 10 (also not shown). The support member 60 may, for example, be made of a polymeric or cellulose-based material possessing the requisite rigidity. Advantageously, given the considerations of both cost and strength, the support member 60 may be made of a material selected from acrylonitrile butadiene styrene (ABS); polyethylene (PE); a pressure-sensitive painted nylon material, such as PSP; polycarbonate (PC); and so on. The support member 60 could be made in any suitable shape, provided it is configured to correctly engage with a suitable medical device. Similarly, the support member 60 may also have any suitable thickness or depth (i.e. the perpendicular distance between the top 61 and bottom 62 surfaces of the support member 60). For example, where a more flexible material is used, the support member 60 may be thicker. However, the volume of the aperture 33 should be taken into consideration. In this regard, the aperture may be made narrower in a thicker support member than it is in a thinner support member to maintain a desired volume for receiving a fluid sample to be tested. However, the aperture size must be designed relative to the detecting system of the medical device, such that the sample contacts at least the minimum surface area of the sample interface (or sample receiving area) 63 of the medical device 10 that is required for an accurate measurement of a property of the sample.

In another embodiment, the support member 60 may comprise a type of filter, membrane, or a porous substrate. In this way it may be possible for the support member 60 to serve the purpose of removing (e.g. by filtration) an undesirable component in the fluid sample; or it may otherwise improve the quality of the sample to be tested, e.g. to reduce noise or improve a signal-to-noise ratio in the detected results.

Referring to FIG. 4B, in a preferred embodiment the bottom surface 62 of the support member 60 is provided with a system (e.g. a wiping system) for removing fluid from the surface of the sample interface 63 of the medical device 10, which may be in the form of resilient pads 42. The system for removing fluid may be any other suitable systems, such as a wiper blade, which serves the purpose of cleaning (in particular removing fluid from) the surface of the sample interface 63 before and/or after the measurement of the sample has been taken. In this way, contamination of the sample (and the measurement) by undesirable contaminants can be avoided or at least reduced. The resilient pads 42 may be constructed from any suitable material, including but not limited to poly(vinyl alcohol); a non-woven material; and cotton fabric. Preferably, the resilient pads 42 are made from poly(vinyl alcohol). Furthermore, the resilient pads 42 could be designed to perform a fluid dynamic function, such as to absorb any fluids in contact with the pads. One or more resilient pads (or other wiping or absorbing systems) may be used. Where only one resilient pad is used it is advantageously disposed on the bottom surface 62 of the support member 60 distal to the aperture 33 (i.e. between the aperture 33 and the distal end 64).

In alternative embodiments (not shown) the support member 60 may be provided with more than one aperture 33 to enable more the one sample to be tested with a single test strip 32. For example, the support member 60 may be provided with two apertures 33, provided that the support member can engage with the medical device 10 in such a way than both apertures 33 can be vertically aligned with the sample interface 63 of the medical device 10.

As previously noted, any shape of aperture 33 can be used, provided it is compatible with the medical device. For example, the aperture 33 may be circular in shape with a diameter of approximately 0.201 inches±0.003 inches (5.1 mm approx.) so that a small droplet of a test sample can quickly fill the aperture 33, and so that the user can easily determine the amount of sample needed.

An alternative embodiment of the test strip 32 of the invention is shown in FIG. 4C. In this embodiment the test strip 32 has a trigger (or tip) 43 at the distal end 64 of the support member 60, which interacts with a sensor means 121 (indicated in FIG. 2) of the medical device 10. The trigger 43 may be capable of mechanical or electrical interaction with the sensor means 121 of the medical device 10, so as to enable the function of the medical device 10; or in certain embodiments, to control a specific function of the medical device. When the trigger 43 is capable of electrical communication with the medical device 10, stored information may provide security details, or provide relevant information on the particular method of the invention to be implemented by the medical device 10. Thus, a test strip 32 may be pre-programmed for use in a method of determining GRV, or in a method for determining the concentration of dietary formula in a sample of fluid, and so on. The trigger 43 may also or alternatively be configured to mechanically push the sensor means 121 (see FIG. 2), which triggers a response in the detecting sample means 20. For instance, the interaction of the trigger 43 with the sensor means 121 may simply provide an indication that a test strip 32 has been placed in the detecting area 31 of the medical device 10.

The trigger 43 may be positioned at any point on the test strip 32, provided it is configured to interact with an appropriately placed sensor means 121 on the medical device 10. Preferably, however, the trigger 43 is located at or towards the distal end 64 of the test strip 32.

As indicated, the sensor means 121 of the medical device 10 could be a mechanical sensor, a photo-interrupt sensor, or any other type of sensor, provided that it is capable of receiving a signal from the trigger 43 of the test strip 32. Advantageously, the sensor means 121 is a photo-interrupt sensor.

Another preferable embodiment of the test strip 32 of the invention is shown in FIG. 4D, in which the top diagram shows the front (top) view of the test strip 32; and the bottom diagram shows the rear (bottom) view of the same test strip 32. This embodiment demonstrates a specifically shaped test strip 32 that is designed with a handle member at the proximal end 65 of the test strip 32 for ease of handling. As in FIG. 4C, a trigger 43 is provided at the distal end 64 of the test strip 32. The handle member 66 may be a broadened extension (or enlarged region) of the support member 60 and may be shaped in an aesthetic or ergonomic manner. In addition, the handle member 66 may be provided with a surface that provides improved handling or grip when held by the user. In the embodiment depicted, the top surface of the handle member 66 is provided with a region of raised spots or dimples 44, and the bottom surface of the handle means 66 is provided with a region of ridges or contoured stripes 46. It will be appreciated, however, that the exact form of the surface is not important, provided that the surface is intended to allow the test strip 32 to be conveniently handled/gripped by the user (or by a mechanical instrument, such as a robotic arm). In addition, or in the alternative the handle member 66 may be formed from or at least partly coated with a material of higher frictional coefficient, such as a rubber material. For example, the dimples 44 or ridges 46 may also be made of a rubber material. Other suitable surfaces may be non-gloss plastics (e.g. roughened plastics) and the like.

The test strip 32 of FIG. 4D is also provided with a sealing system in the form of a resilient ring 45, which is disposed about the aperture 33 on the bottom surface 62 of the support member 60. The resilient ring 45 may be made of any suitable material provided it can function as a seal between the aperture 33 and the sample interface 63 of the medical device 10, in use. For example, the resilient ring 45 is beneficially water-proof and may be made by polymers of ethylene-vinyl acetate (EVA), rubber, and/or silica gel, and/or combination thereof. It will be appreciated that the sealing system need not be in the form of a ring, for example, a circle or square of a resilient material can equally serve the intended purpose, provided that the material has a hole that in shape corresponds to the rim of the aperture 33 on the bottom surface 62 of the support member 60.

It should be noted that where the aperture 33 has a circular opening, the aperture 33 through the support member 60 may be cylindrical or frustoconical. In one embodiment the aperture 33 in the support member 60 is frustoconical. Where the aperture 33 is frustoconical it is advantageous for the larger opening to be in the upper surface 61 of the support member 60.

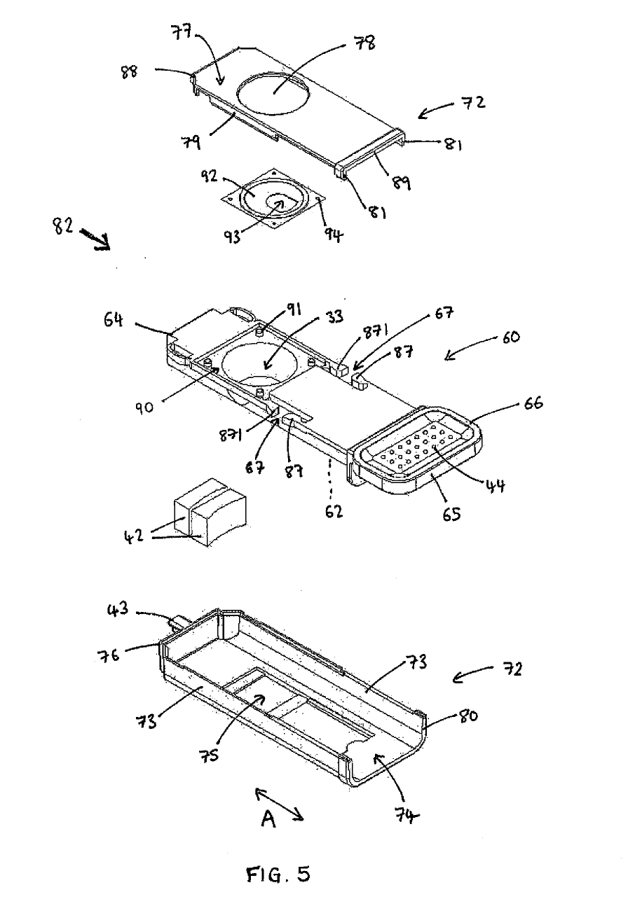

FIG. 5 shows an exploded perspective view of a preferred embodiment of a test strip 82 comprising a support member 60 that is slidable within a sleeve 72. In this embodiment, a support member 60 is elongate, having a distal end 64 and a proximal end 65. The support member 60 is provided with an aperture 33 extending from the top surface 61 to the bottom surface 62 (not shown) of the support member 60. At the proximal end 65 of the support member is a handle means 66, with a textured (dimpled) surface 44 as previously described (see reference to FIG. 4D). A pair of resilient pads 42 is disposed on the bottom surface 62 of the support member 60 distal to the aperture 33. In addition, the support member 60 is provided with lateral engagement (or locking) members 67 for engaging with the sleeve 72. The lateral engagement members 67 comprise, in one embodiment, opposing pairs of resilient arms (or teeth) 87, 871, which lie in the plane of the support member 60 and flex elastically from the attachment point to the support member 60 (i.e. at the shoulder) in the plane of the support member 60. In the absence of a resistive force each arm 87, 871 tends to flex laterally away from the edge of the support member 60. The proximal arm 87 of each opposing pair is directed substantially distally, with its shoulder at the proximal end of the arm 87; while the opposing tooth 871 of each pair is positioned distally to the tooth 87, and is directed towards the proximal end 65 of the support member 60 with its shoulder distal to the arm. There are two such engagement members 67, which may be provided approximately midway between the distal end 64 and the proximal end 65 of the support member 60 and are arranged with one engagement member 67 at each lateral edge of the support member 60. The operation of the engagement members 67 will be described later.

As in the previous embodiments described, the bottom surface 62 of the support member 60 may be provided with a sealing system (not shown), such as a water-proof resilient ring, around the aperture 33, to prevent fluid leaking from the aperture 33, in use. Advantageously, the aperture 33 extends from the top surface 61 to the bottom surface 62 of the support member 60. However, as previously described with respect to the aperture 33 of the test strip 32 depicted in FIG. 3A, the bottom of the aperture 33 may alternatively be covered with a layer (or lens) of a suitable transparent material, such as glass, quartz or plastic (not shown), to produce a water-tight container for the fluid sample.

The test strip 82 further comprises a sleeve 72, which comprises an upper section 77 and a lower section 74, and is adapted to enclose (or surround) at least a portion of the support member 60. The lower section 74 of the sleeve 72 is provided with a window 75 and upwardly extending lateral flanges 73, which may engage with the lateral edges of the support member 60. In the embodiment depicted the lower section 74 of the sleeve 72 further comprises an upwardly extending distal flange 76, which is provided with a trigger 43 as previously described. However, in some embodiments the lower section 74 of the sleeve 72 does not comprise an upwardly extending distal flange. In such embodiments, when a trigger 43 is required, the trigger 43 may be positioned at the distal end 64 of the support member 60.

The upper section 77 of the sleeve 72 is also provided with a window 78 and downwardly extending side flanges 79, which may be positioned towards the distal end 88 of the upper section 77 of the sleeve 72, and downwardly extending members 81 at the proximal end 89 of the upper section Advantageously, as depicted, the downwardly extending side flanges 79 and downwardly extending members 81 do not extend along the entire length of the lateral edges of the upper section 77; i.e. they do not form a continuous lateral wall. Conveniently, the downwardly extending side flanges 79 and/or downwardly extending members 81 may provide for a system of engaging with the lower section 74 of the sleeve 72. Any suitable system of engagement between the upper and lower sections 77 and 74, respectively, of the sleeve 72 may be used; for instance, an adhesive or a mechanical connection may be employed. A suitable means of engagement is a snap-fit mechanism.

The lower section of the sleeve 74 is configured to engage with the support member 60 in such as way that the window 75 is in alignment with the aperture 33. Typically, the window 75 is at least as large as the aperture 33 in the bottom surface 62 of the support member 60, so that, in use, the aperture 33 is not obscured from the sample interface 63 of the medical device 10. Accordingly, the window 75 may be the same size and shape as the aperture 33. Advantageously, as depicted, the window 75 is slightly larger than the aperture 33. In this case, the window 75 need not be the same shape as the opening of the aperture 33, for instance, the aperture 33 may be circular and the window 75 may be rectangular (as depicted) or square.

Furthermore, the upper section 77 of the sleeve 72 is adapted to engage with the lower section 74 of the sleeve 72 such that the window 78 in the upper section 77 is in alignment (i.e. vertically aligned) with the window 75 in the lower section 74 of the sleeve 72. Moreover, the sleeve 72 is configured to engage with the support member 60 such that the window 78 in the upper section 77 of the sleeve 72 is aligned with the aperture 33 in the top surface 61 of the support member 60. However, it is not necessary that the window 78 in the upper section 77 of the sleeve 72 is the same size and shape as the window 75 in the lower section 74 of the sleeve 72. For example, the window 78 may be larger than, the same size as, or even smaller than the aperture 33 in the top surface 61 of the support member 60.

It should be appreciated that in embodiments of the invention that comprise a sleeve 72, the upper section 77 of the sleeve 72 may be an optional feature. In embodiments that do not comprise an upper section 77, the upwardly extending lateral flanges 73 of the lower section 74 of the sleeve 72 are preferably adapted to mechanically grip the support member 60. For example the upwardly extending lateral flanges 73 may turn inwardly to enclose at least a portion of the top surface of the support member 60. Alternatively, the lateral sides of the support member 60 and the inwardly facing sides of the upwardly extending lateral flanges 73 may be adapted for mutual engagement, for example, in a form of guide rail.

The support member 60 is preferably (as depicted) a slide fit within the sleeve 72, so as to allow longitudinal movement of the support member 60 relative to the sleeve 72, as indicated by arrow A, but to substantially prevent lateral movement. Therefore, the lower section 74 of the sleeve 72, and in particular the upwardly extending lateral flanges 73 are adapted to constrain the lateral edges of the support member 60, such that the upwardly extending lateral flanges 73 act to guide longitudinal movement of the support member 60, while substantially preventing lateral movement.

The test strip 82 of the preferred embodiment depicted further comprises a lid 92 (or cover) to cover at least a portion of the aperture 33 in the top surface 61 of the support member 60. The lid 92 is advantageously adapted to engage with the top surface 61 of the support member 60, as depicted. In this embodiment, the top surface 61 of the support member 60 has four small projections 91 arranged in a square about the aperture 33. In addition, or in the alternative, the top surface 61 of the support member 60 may be provided with a recessed area 90, for example, in the form of a recessed square, around the aperture, into which the lid may locate. Advantageously, as depicted, the lid 92 may be in the form of a thin square having a small hole 94 towards each corner that is positioned to locate onto each of the corresponding small projections 91 on the top surface 61 of the support member 60, thereby to fix the position of the lid 91 relative to the aperture 33.

The lid 91 may be provided with an opening 93 through which a sample of fluid can be passed to fill the aperture 33. It will be appreciated that the lid 91 serves the purpose of reducing the amount of external light entering the aperture 33, which could distort the measurements of the medical device 10; and also serve the purpose of reducing the chance of spilling the contents of the aperture 33 and, for example, contaminating the medical device 10. As such, the lid 91 need not be positioned between the upper section 77 of the sleeve 72 and the top surface 61 of the support member 60. Thus, in alternative embodiments the lid 91 may be located above the upper section 77 of the sleeve 72, and need not have an opening 93. For example, the lid 91 may be hinged to the upper section 77 of the sleeve 72 or it may be removable, to enable a sample of fluid to be placed into the aperture 33.

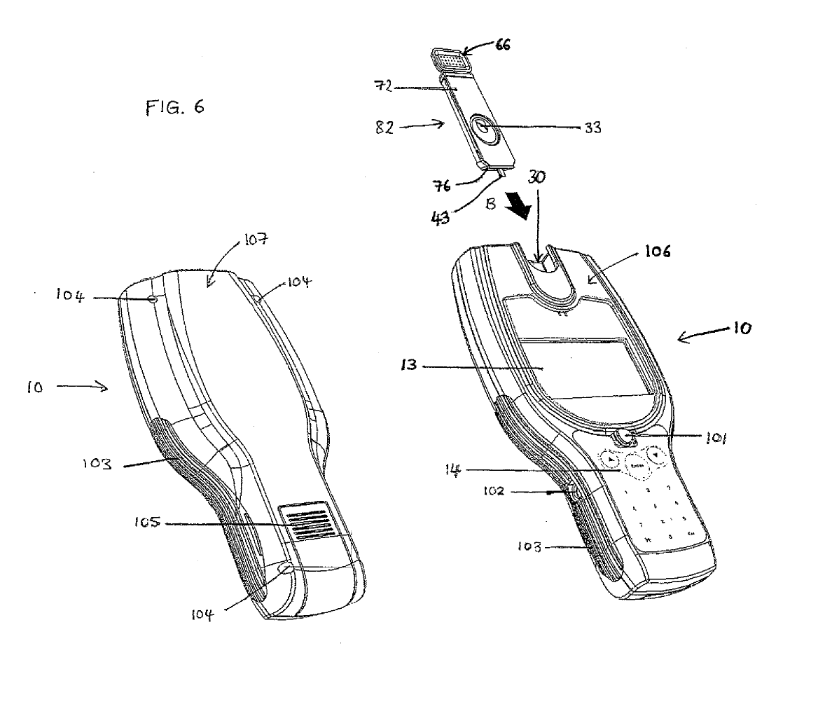

In use, the test strip 82 is typically arranged such that the windows 75 and 78 in the sleeve are aligned with the aperture 33 in the support member 60. This can be considered to be the first position of the support member 60 relative to the sleeve 72. In this first position, a sample of fluid can be conveniently loaded into the aperture 33 through the window 78 in the upper section 77 of the sleeve 72 and the opening 93 in the lid 92. Once the test strip 82 has been inserted into a medical device 10 and a measurement of a parameter of the fluid has been taken (as will be described with reference to FIG. 6), it is advantageous to enclose the fluid sample in the sleeve 72 for disposal. To enclose the fluid sample the aperture 33 is moved out of alignment with the windows 75 and 78 (and the opening 93), by sliding the support member 60 away from the distal end 76, 88 of the sleeve 72. When the support member 60 is slid longitudinally within the sleeve 72 away from the upwardly extending distal flange 76 of the lower section 74 of the sleeve 72, the resilient arms 87, 871 of the engagement members 67 are held against (or towards) each lateral edge of the support member 60 by the downwardly extending side flanges 79 of the upper section 77 of the sleeve 72. However, once the opposing arms 87, 871 have slid beyond the proximal ends of the downwardly extending side flanges 79, there is no lateral restraint and the arms 87, 871 are able to flex in the plane of the support member 60 laterally away from the edges of the support member 60. As the longitudinal movement of the support member 60 continues, eventually the proximal arm 87 of each opposing pair contacts the downwardly extending member 81 at the proximal end 89 of the upper section 77 of the sleeve 72. The shoulder of each proximal arm 87 rides past the downwardly extending members 81 and each proximal arm 87 is pushed laterally towards the support member 60. Once the proximal arm 87 has slid fully past the downwardly extending member 81, it can spring back to its position away from the lateral edges of the support member 60. If the support member 60 continues to slide out of the sleeve 72 the distal arm 871 comes into contact with the downwardly extending member 81. However, since the distal arm 871 points proximally with its shoulder distal to the arm, the arm 871 does not ride under the downwardly extending member 81 and instead it is forced laterally outwards against the downwardly extending member 81 until a stop is reached (i.e. the support member 60 is unable to slide further proximally, because the downwardly extending member 81 cannot move beyond the inside edge of the shoulder of the distal arm 871). Likewise, any sliding movement of the support member 60 back into the sleeve 72 is substantially prevented, because the proximal arm 87 has already travelled fully past the downwardly extending member 81. Hence, as with the distal arm 871, it cannot be slid underneath the downwardly extending member 81 as the arm 87 and not its shoulder is now pointed towards the downwardly extending member 81.

Accordingly, the support member 60 is engaged (or locked) into a second position relative to the sleeve 72. In this second position the aperture 33 has moved fully within the sleeve 72 and the fluid sample is, therefore, enclosed within the sleeve.

It will be appreciated that in this embodiment the strength of resilience of the arms 87, 871 may determine the reversibility of the second position of the support member 60. In the embodiment depicted it is possible to manually depress the arms 87, 871 towards the lateral sides of the support member 60 in order to allow the arm concerned to be slide past the downwardly extending member 81; for example, in order to slide the support member 60 back into the first position, or to remove the support member 60 fully from the sleeve.

Turning to FIG. 6, a preferred medical system according to the invention comprises a test strip 82 as described in relation to FIG. 5 and a medical device 10 in the form of a hand-held refractometer.

The back of the hand-held refractometer 10 is shown in the left-hand diagram and the front of the hand-held refractometer is shown in the right-hand diagram with a test strip 82 of the invention ready to be inserted into the channel 30 of the refractometer 10. The hand-held refractometer 10 may comprise a front section 106 and a back section 107 that are connected to each other, for example, using screws as indicated at screw holes 104. Of course, any suitable mechanism for assembling the hand-held refractometer 10 can be employed. The back section 107 of the hand-held refractometer 10 further comprises a battery compartment 105, which may be suitable for either rechargeable or non-rechargeable batteries.

As indicated in the front view of the hand-held refractometer 10, the front section 106 has a display 13, a keypad 14, and a channel 30 for receiving a test strip 82, as previously described. In one embodiment the hand-held refractometer 10 further comprises a power (i.e. on/off) button 101 for switching the hand-held refractometer 10 on and off; and a DC adapted socket 102 for receiving a power source, either to charge the batteries (if rechargeable batteries are used) or for directly powering the hand-held refractometer 10. In addition, the hand-held refractometer 10 is provided with gripping members, for example, in the form of rubber panels 103 along the lateral edges of the hand-held refractometer 10. The rubber panels 103 serve to improve the users grip on the hand-held refractometer 10.

In use, the medical device 10 is typically first turned on (e.g. by pressing button 101), for example, to allow it to warm up prior to the time at which a measurement of a parameter of a fluid sample is to be taken. Next any calibrations that may be necessary before a test sample (e.g. a gastric content fluid) can be measured are carried out. When the device is ready for use an appropriate test strip 82 in accordance with the invention (and as described with reference to FIG. 5) is selected for use.

A suitable volume (e.g. 0.4 to 2.0 ml) of a sample fluid to be measured by the medical device 10 is placed in the aperture 33 of the test strip 82. The fluid sample may be placed into the aperture 33 using any suitable method, such as using a pipette. The test strip 82, held by the handle member 66 is then inserted into the channel 30 of the medical device 10, for example, by sliding in the direction of arrow B. The medical device 10 is preferably a refractometer and comprises a sample interface (not visible in FIG. 6), at which a property of the fluid sample is measured. At least the distal end of the test strip 82 (i.e. at least the upwardly extending distal flange 76 of the sleeve 72 and the trigger 43) engages with the channel 30 of the medical device 10, and the test strip 82 continues to slide along the channel 30 until the upwardly extending distal flange 76 of the sleeve 72 and the trigger 43 contact the end of the channel 30. At this point the trigger 43 communicates with the sensor means 121 within the medical device 10, for example, to indicate that a test sample is in place. Also at this point, due to the configuration of the test strip 82 and the channel 30, the bottom of the aperture 33 is directly aligned with the sample interface 63 (not visible) of the medical device 10.

The engagement (or interaction) between the channel 30 and the test strip 82 is configured such that a compressive force is exerted across the test strip 82 in the direction perpendicular to the plane of the sample interface 63 (i.e. between the upper section 77 and the lower section 74 of the sleeve 72). In this way, the bottom surface 62 of the support member 60 (optionally including a resilient ring 45) is compressed against the sample interface 63 of the medical device 10, to help prevent leakage of fluid from the aperture 33. The interaction between the (sides of the) channel 30 and the test strip 82 should, therefore, be sufficiently tight to exert a suitable compressive force on the test strip 82, but should not be so tight that the test strip cannot be inserted, or is broken under the force exerted. In embodiments that have an open channel rather than an enclosed channel; for instance, as depicted in FIG. 3A, it may be advantageous to push the test strip firmly but carefully against the channel surface (i.e. by providing a force in the direction of the sample interface 63), in order to improve the seal between the aperture 33 and the sample interface 63.

Next, the medical device 10 takes a measurement of a parameter of the fluid sample and, typically, reports the measurement on the display means 13 of the medical device 10. It is generally not necessary to manually record the measurement, because it will be stored in the memory of the medical device 10. Before the test strip 82 is withdrawn (or removed) from the channel 30 of the medical device 10, the fluid sample is preferably enclosed (stored or housed) within the sleeve 72 of the test strip 82. Accordingly, the support member 60 is slid longitudinally out of the sleeve 72, bringing the aperture 33 out of alignment with the window 75 in the lower section 74 of the sleeve 72. In so doing, the fluid sample is removed from the sample interface 63 and enclosed within the sleeve 72. The bottom surface 62 of the support member 60 may be provided with a water-proof, resilient ring 45 to prevent (or at least reduce) leakage of fluid from the aperture 33 onto the sample interface 63.

As the support member 60 is slid out of the sleeve 72, the resilient pads 42 are brought into alignment with the window 75 of the lower section 74 of the sleeve 72; and thereby, into contact with the sample interface 63 of the medical device 10. The sleeve 72 (or in some embodiments the channel 30 of the medical device 10) is configured so that the resilient pads 42 are compressed. Therefore, as the resilient pads 42 slide across the surface of the sample interface 63, any residual fluid is wiped from the surface of the sample interface 63. Advantageously, the resilient pads 42 are also absorbent to help remove any residual fluid. As the movement of the support member 60 continues, the aperture 33 and eventually the resilient pads 42 slide past the window 75 in the lower section 74 of the sleeve 72 and are then enclosed in the sleeve 72. Finally, the support member 60 is slid into it second position (as previously described), in which longitudinal movement in either direction is substantially prevented by an engagement system 67. At this point continued pulling on the handle means 66 (or on the sleeve 72 directly) may slide the test strip 82 out of the channel 30 and the test strip is thereby fully removed from the medical device 10. Preferably, the test strip 82 is disposable and it is then discarded.

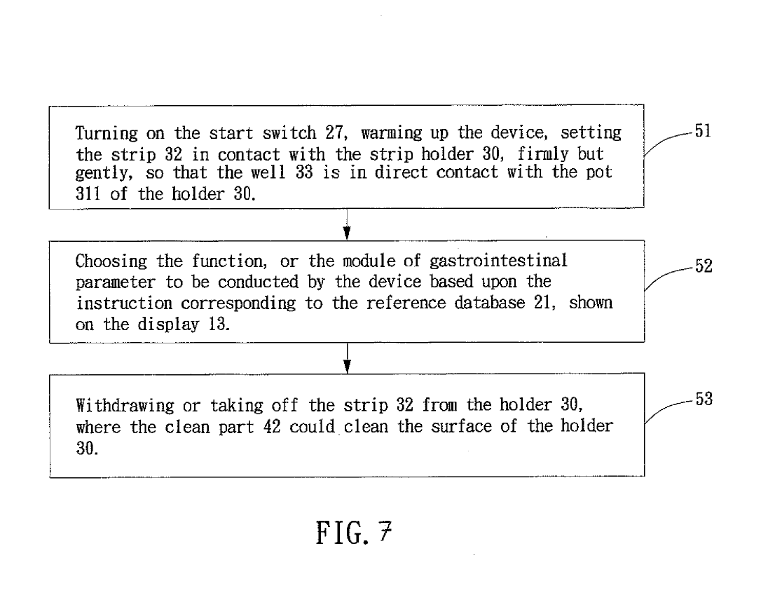

FIG. 7 provides a flow chart summarising an embodiment of the overall process employing a medical system 70 and methods according to the invention. In particular, step 51 relates to steps taken before a measurement of a sample fluid is taken. These steps involves turning on the start switch 27 of the medical device 10, allowing the medical device 10 to warm up, and conducting a calibration, or any other pre-conditions that may be necessary. The test strip 32 is placed in contact with the surface of the channel 30. It may be advantageous to press the test strip 32 firmly but carefully against the surface of the channel 30, so that the aperture 33 is brought into direct contact with the sample receiving means (or sample interface 63) of the medical device 10. As previously mentioned, the test strip 32 may be adapted to be responsive to at least one fluid sample or application of the medical device 10, and the detecting sample means 20 of the medical device 10 may be pre-programmed or instructed (by the user or by information contained in the test strip 32) to measure and determine one parameter in the sample fluid, such as the soluble solid content (refractive index or Brix value) of the fluid.

Step 52 relates to the taking of a measurement of a fluid sample. It involves choosing the required function to be conducted by the medical device 10 (such as the measurement of a particular gastrointestinal parameter). Any measurements of the fluid sample taken are related to corresponding values in the reference database 21, to determine the required parameter, and the result is shown on the display 13.

Step 53 relates to the stages that may take place once the medical device 10 has made a measurement of a fluid sample. First the test strip 32 is withdrawn or removed from the channel 30. Advantageously, a wiping system simultaneously cleans (e.g. removes fluid from) the sample interface 63 of the channel 30. In one embodiment, a hard copy (print-out) of the measurements and/or calculations made by the medical device 10 are enabled, for example, by means of a docking station 34 and a paper exit 35.

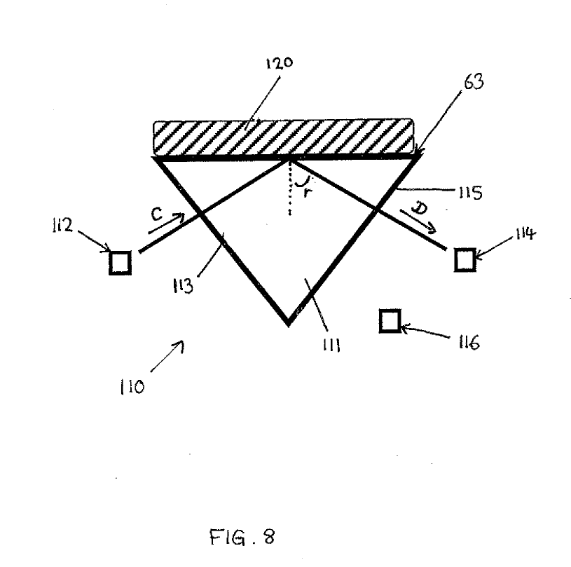

FIG. 8 is a schematic representation of a preferred optical module for use in a medical device according to the invention.

The optical module 110 comprises a prism 111 (which is substantially transparent) having a sample interface 63 which, in use, contacts a fluid sample 120. Advantageously, the prism 111 is made of glass, quartz or a plastics material. The material used may be selected, for example, on the basis of cost, required quality and intended use. A light source in the form of a laser diode 112 is arranged to project light through a first face 113 of the prism 111 onto the underside of the sample interface 63, as indicated by arrow C. The laser diode 112 provides a number of advantages over the conventional use of light emitting diode (LED) light sources. For example, the laser diode 112 provides a directional source of light energy, which reduces the noise in the reading system and does not require an accurate baffle to cut out a large proportion of the light energy. The laser diode 112 may be set at any suitable wavelength and is typically from 600 to 1300 nm, beneficially from 650 to 850 nm and most advantageously from 700 to 800 nm. The laser diode is typically arranged to project light onto the sample interface at an angle of incidence of 42 to 62°, from 50 to 62° and advantageously from 55 to 62°.

The optical module 110 further comprises an array of photoreceptors, which are arranged adjacent to a second face 115 of the prism 111 to detect light that is reflected from the sample interface 63 (when a fluid sample 120 is present), as indicated by arrow D, at an angle of reflection (r), which is dependent on the difference between the refractive index of the prism and the refractive index of the fluid sample 120. The array of photoreceptors may be in the form of one or more photodiodes, for example, 1, 2, 3 or 4 photodiodes arranged in an array. In one embodiment the optical module comprises a first photodiode 114 arranged adjacent to the second face 115 of the prism to detect any reflected light. A further advantage of the use of a laser diode 112 is that a photodiode can be readily used to detect reflected light and, therefore, to measure the refractive index of a sample fluid. In contrast the prior art systems that used LED light sources typically employ an far more expensive charge-coupled device (CCD) to collect and analyse detected light, which also requires complicated algorithms to determine the most intense angle of reflected light and to resolve any noise.

In a preferred embodiment the optical module 110 further comprises a second photodiode 116 also positioned adjacent to the second face 115 of the prism 111. The second photodiode is positioned at a suitable angle to detect light that may be scattered, rather than refracted, from the fluid sample 120. In this way, the optical module 110 can advantageously be employed to measure the refractive index (and BV) of a heterogeneous fluid, such as one that comprises an emulsion or suspension. In contrast, an optical module 110 having only one photodiode 114 is more suitable for measuring homogeneous solution fluid samples. Medical devices 10 in accordance with the invention, which comprise an optical module 110 having two photodiodes 114, 116 (as indicated in FIG. 8) provide a particular advantage over the prior art, because they can be used, for example, to accurately measure parameters of gastric content samples and dietary formulas which may contain suspensions or emulsions. A further advantage of the optical module 110 having two photodiodes 114, 116 is that a refractometer containing the optical module can be programmed to provide a double-check of each reading made by the device by using each of the photodiodes, one reading reflected light and one measuring scattered light. The optical modules of the invention are particularly advantageous over the prior art for accurately measuring solutions of low dietary formula concentrations (e.g. 0 to 30%).

To assist in understanding the present application, the following examples are included and describe the results of a series of experiments carried out using the methods and medical systems or devices of the invention. The following examples relating to this application should not be construed to specifically limit the application or such variations of the application, now known or later developed, which fall within the scope of the application as described and claimed herein.

The entire contents of the patents and other references cited herein are hereby incorporated in this patent application in their entirety.

### EXAMPLES

The following examples illustrate methods of preparing, characterising, and using the methods, devices and systems of the present invention. These examples are in no way intended to limit the scope of the invention.

### Example 1

Use of Brix Value to Monitor Dietary Formula Concentration

Materials and Methods

Brix values for nutrients such as minerals, vitamins mixtures, carbohydrate, protein, fat, and polymeric dietary were determined with the device or system according to this invention. A solution of minerals (Ringer's solution) was obtained from YF Chemical Corporation (Taipei, Taiwan), and consisted of is sodium chloride (8.6 mg/ml), potassium chloride (0.3 mg/ml), and calcium chloride (0.33 mg/ml). Vitamins (Lyo-povigen, a parenteral vitamin mixture) was also obtained from YF Chemical Corporation (Taiwan), and contained vitamin A palmitate (12 IU/ml), vitamin D2 (1 IU/ml), vitamin E (0.005 IU/ml), vitamin C (0.5 mg/ml), vitamin B1 (0.05 mg/ml), vitamin B2 (0.01 mg/ml), vitamin B6 (0.015 mg/ml), niacinamide (0.1 mg/ml), and d-panthenol (0.025 mg/ml). Carbohydrate (Carb-aid, Corn starch) and protein (Whey-aid, lactoalbumin) were purchased from Nutritec-Enjoy Nutrition Center, Taiwan. Fat (Intralipid) was purchased from Frenius Kabi AB, Uppsala, Sweden. Full strength polymeric dietary (Osmolite HN, Ross, Ohio, USA) contained carbohydrate (17 g/100 ml), protein (5.3 g/100 ml), and fat (4.1 g/100 ml).

The Brix values were measured using the device or system according to this invention, whose Brix scale (% Brix) of 0-50 could be read in 0.2 increments, further, the scale being improved to 0-95, in 0.01 increments is designed and manufactured. These results were close to the inventor's previous invention as disclosed in the pending application Ser. No. 10/787,705, using a conventional refractometer (N.O.W. 507-1, Nippon Optical Works; Tokyo, Japan). To measure the solute concentration, one or two drops, or more, of the specimen fluid were placed in a designated well in the strip of the apparatus according to this invention. The detecting unit of the device according to this invention was calibrated with distilled water before each measurement.

Statistical Analysis

Results are presented as the mean±SEM. Correlation coefficients following linear regression analysis were used to evaluate the relationship between Brix values and dietary formula concentrations. Differences were considered statistically significant when P<0.05.

Results

Brix Values of Nutrients

The Brix values of nutrients and dietary formula are measured as follows: Distilled water, minerals, and vitamins contained little dissolved material and had correspondingly low Brix values of 0±0, 1.4±0.2, and 0.5±0.2, respectively. Carbohydrate (17 g/100 ml), protein (5.3 g/100 ml), fat (4.1 g/100 ml), and full-strength polymeric dietary had high concentrations of dissolved nutrients and correspondingly high Brix values of 12.4±0.5, 6.2±0.3, 5.9±0.3, and 22.9±0.4, respectively.

Molar Refractivities in Solution of Mixed Nutrients Were Additive. Brix values (Brix %) of pure nutrients (such as carbohydrate, protein, and fat) and mixtures of these were measured. Three dilutions (50%, 100% and 200% of the starting concentration) of carbohydrate, protein, and fat were made with distilled water. The Brix value was a linear additive function of the solute concentration, regardless of whether the solute was carbohydrate, protein, or fat.

The results presented are mean±SD. They are as follows: 8.9 g/100 ml, 17.3 g/100 ml, and 33.6 g/100 ml of carbohydrate are 6.2±0.7, 12.8±0.2, and 26.1±0.3, respectively; 2.8 g/100 ml, 5.1 g/100 ml, and 10.8 g/100 ml of protein are 3.2±0.1, 6.1±0.3, and 11.6±0.2, respectively; and 2.4 g/100 ml, 4.4 g/100 ml, and 8.1 g/100 ml of fat are 3.3±0.1, 6.2±0.3, and 11.6±0.4, respectively. More, the Brix value of the nutrient including carbohydrate (16.8 g/100 ml) and protein (5.6 g/100 ml) is 18.5±0.8; and the Brix value of the nutrient including carbohydrate (16.8 g/100 ml), protein (5.5 g/100 ml), and fat (4.6 g/100 ml) is 23.2±0.3.

For nutrient solutions, consisting of some combination of carbohydrate (17 g/100 ml), protein (5.3 g/100 ml), and fat (4.1 g/100 ml), Brix value was also an additive function of the component concentration. For example, a mixture of carbohydrate (17 g/100 ml) and protein (5.3 g/100 ml) had a Brix value of 20.1±0.5, and a mixture of carbohydrate (17 g/100 ml), protein (5.3 g/100 ml), and fat (4.1 g/100 ml) had a Brix value of 24.1±0.6, which was close to the Brix value (23.5±0.1) of full-strength polymeric diet.

Brix values of polymeric dietary in fasting gastric juice. Table 1 shows the effect of gastric juice dilution on the Brix value of the polymeric dietary formula. The Brix value measurements of polymeric dietary in fasting gastric juice were made at 5, 30, 120 and 240 min, respectively. Brix values correlated with the concentration of polymeric dietary diluted in fasting gastric juice at each time with minimal variability (R2<0.98). Therefore, polymeric dietary formula concentration in gastric contents can be estimated on the basis of the linear regression equation; Full strength polymeric diet % concentration=Brix value÷0.23, wherein 0.23 is the slope of serially diluted dietary formula concentration. The range of slope can be from 021 to 0.25, based upon the circumstances of the correlated test. In this example, for example, a Brix value of 6.1 and 12.1 in gastric contents corresponds to a 25% and 50% full-strength polymeric dietary concentration, expressed as % concentration of dietary formula.

TABLE 1
Brix values of polymeric diet in gastric juice
% Full-strength polymeric diet

| Time | | | | | | | |
|---|---|---|---|---|---|---|---|
| (min) | 0 | 12.5 | 25 | 50 | 75 | 100 | R² |
| 5 | 2.2 ± 0.4 | 4.5 ± 0.4 | 6.7 ± 0.5 | 12.5 ± 0.8 | 17.8 ± 0.4 | 23.6 ± 0.2 | 0.99 |
| 30 | 2.3 ± 0.2 | 4.7 ± 0.3 | 6.5 ± 0.8 | 12.4 ± 0.9 | 17.9 ± 0.3 | 23.1 ± 0.3 | 0.98 |
| 120 | 2.1 ± 0.5 | 4.5 ± 0.5 | 6.8 ± 0.7 | 12.2 ± 0.8 | 18.0 ± 0.3 | 23.4 ± 0.2 | 0.98 |
| 240 | 2.1 ± 0.5 | 4.6 ± 0.6 | 6.8 ± 0.8 | 12.4 ± 0.5 | 17.8 ± 0.5 | 23.5 ± 0.4 | 0.98 |

Brix values (% Brix) of polymeric diet (Osmolite HN) diluted in fasting gastric juice. Results are presented as means ± SEM (n = 3).

### Example 2

Monitoring Bolus Nasogastric Feeding by the Brix Value Determination of Gastric Contents and Residual Volume Measurement of Gastric Contents

Materials and Methods

All BV measurements were done by using the system according to this invention, whose Brix scale (% Brix) of 0-50 could be read in 0.2 increments, further, the scale being improved to 0-95, in 0.01 increments is designed and manufactured. The device or system according to this invention was calibrated with distilled water before each measurement. One or two drops, or more, of the test sample, such as specimen fluid, were placed on a designated well in the strip of the device according to this invention for observation, all measurements made at room temperature using natural light. In this way, the concentration of soluble solids in solution was measured at the bedside for each specimen.

Brix values for a polymeric (Osmolite HN, Abbott Laboratories, Columbus, Ohio) and five solutions (distilled water, 0.9% sodium chloride, 5% dextrose, fasting saliva, and gastric juice) were determined with the refractometer. Each liquid was evaluated six times. Serial dilutions of the polymeric formula (100%, 50%, 25%, 12.5%, 6.2%, and 0%) were made with three of the solutions (distilled water, saliva, and gastric juice) and is shown in the table below. The Brix value was measured in vitro, again performing six separate evaluations for each dilution of the polymeric formula. Again, these results were close to the inventor's previous invention as disclosed in the pending application Ser. No. 10/787,705, using a conventional refractometer (N.O.W. 507-1, Nippon Optical Works; Tokyo, Japan).

The Brix Values for the polymeric diet diluted with different solutions in vitro
% Full strength of polymeric diet (Osmolite HN)

| Solution | 0 | 6.2 | 12.5 | 25 | 50 | 100 |
|---|---|---|---|---|---|---|
| Distilled water | 0 ± 0 | 2.5 ± 0.1 | 3.6 ± 0.2 | 6.5 ± 0.4 | 12.6 ± 0.2 | 23.4 ± 0.1 |
| Saliva | 1.6 ± 0.1 | 2.4 ± 0.4 | 4.7 ± 0.3 | 7.8 ± 0.2 | 12.1 ± 1.2 | 23.5 ± 0.2 |
| Gastric juice | 1.8 ± 0.7 | 5.0 ± 0.8 | 5.7 ± 1.0 | 8.1 ± 1.1 | 13.5 ± 1.2 | 22.7 ± 0.9 |

Results are presented as mean values for six separate determinations ± SEM (n = 3).

Patients receiving bolus nasogastric feeding were used in this study. All subjects were fed the full strength polymeric dietary via a 14 French nasogastric feeding tube. The polymeric dietary composition was 16.7% protein, 54.3% carbohydrate, and 29.0% lipid. Caloric requirements were calculated using the Harris-Benedict equation. A total of 250 ml of the polymeric dietary was administered by bolus infusion every 3-6 hours in the 24 hours prior to the study. Aspirated gastric residual volumes were obtained before each bolus feed by aspiration of the feeding tube. Aspirated gastric residual volumes were obtained first in the supine position, and then in the right lateral decubitus position. Following this preliminary period of monitoring, patients were arbitrarily divided into two groups based on conventional use of aspirated gastric residual volume; patients with low gastric residual volumes (<75 ml) were placed in Group 1, and patients with higher gastric residual volumes (>75 ml on at least two occasions) were placed in Group 2. In total, there were 25 subjects in Group 1 (age: range=59-84, mean±SD=75.9±6.6 years) and 18 patients in Group 2 (age: range=44-79, mean±SD=70.6±10.6 years).

After overnight fasting, all remaining gastric juice was aspirated from the stomach via the nasogastric tube using a 60 ml syringe. Then, all subjects received bolus infusion of 250 ml of the polymeric diet. Immediately after feeding, an attempt was made to thoroughly mix the food content in the stomach by aspirating and reinfusing of the nasogastric tube three times with a 60 ml syringe.

Sequential Brix value determinations were made on 2 ml samples of gastric contents at 0, 30, 60, 120, and 180 minutes intervals. At 180 minutes, any fluid remaining in the stomach was aspirated. Its volume was recorded as the aspirate gastric residual volume (Asp GRV), the Brix value measurement made, (pre-diluted BV) and then the contents were reinstilled into the stomach. An added dilutional 30 ml volume of distilled water was infused via the nasogastric tube. The stomach contents again were thoroughly mixed, and a 2 ml sample was obtained for a final BV measurement (Post Dilute BV). The Calculated GRV was determined using the equation. Calculated GRV times PreDilute BV (Calculated GRV+30 ml), and times PostDilute BV. Specific volume of formula remaining at 180 minutes was defined by 2 equations; % Concentration=BV180 min/0.24, and Volume sub formula=% Concentration times Calculated GRV.

The calculated GRV was determined by the equation: Calc. GRV=(30 mi. Pre-Diluted BV)/(Pre-Diluted B−Post-Diluted BV)

Results

Results are presented as mean values±SEM, These results were close to the inventor's previous invention as disclosed in the pending application Ser. No. 10/787,705, using a conventional refractometer (N.O.W. 507-1, Nippon Optical Works; Tokyo, Japan). The Student's t test was used to assess differences in results between patients in Group 1 and Group 2. A p value of less than 0.05 was considered to be statistically significant.

Immediately following the 250 mL polymeric dietary bolus feeding, the mean Brix values, (BVs), of the gastric contents in vivo were shown to be lower both in Group 1 and Group 2 (20.4±0.6 versus 20.9±0.5, respectively) than the mean values for the polymeric formula made in vitro (23.5±0.5).

The serial changes in Brix values for gastric content for the two groups following the bolus polymeric dietary feeding are measured and analyzed (detailed result not shown). Mean serial Brix value measurements decreased in both groups after bolus feeding. For patients in Group 2, the decrease was less, such that at 180 minutes patients in Group 2 had a significantly higher mean Brix value for gastric contents than those patients in Group 1 (10.6±0.2 versus 5.6±0.4, respectively, p<0.01). The measured and analyzed results also show that the aspirated gastric residual volume at 180 minutes was significantly higher for patients in Group 2 than for those in Group 1 (74±10 versus 19±6 mL, respectively, p<0.01).

However, conventional use of GRV obtained by aspiration via a syringe may be inaccurate and unreliable in measuring true volume of contents present in the stomach at any given time. The dilution technique (determining BVs before and after addition of a known volume of water, e.g., 30 ml of distilled water) takes advantage of the relationship between the % Concentration of formula at any dilution and the measured BVs, and may be used to calculate the true volume of contents and the specific volume of formula remaining in the stomach. It is empiric that while an absolute amount of formula in the stomach does not change with the added 30 ml volume of distilled water, the total volume of gastric contents increases while the % concentration of formula and the corresponding BV decreases. The original volume of gastric contents presented in the stomach before dilution (volume unknown) was derived from the change in the BV following dilution. The amount of formula remains constant through dilution and is described by equation: (PreDilute % Conc)×Volumeunknown=(PostDilute % Conc)×(Volumeunknown+30 ml). Substituting the product BV/0.24 for % Concentration generates equation: (PreDilute (BV/0.24))×Volumeunknown=(PostDilute (BV/0.24))×(Volumeunknown+30 ml), both sides of which may be multiplied by 0.24 to derive equation: (PreDilute BV)×Volumeunknown=(PostDilute BV)×(Volumeunknown+30 ml). Solving for the volume unknown volume produces equation: Volumeunknown=((PostDilute BV)×30 ml)/(PreDilute BV−PostDilute BV), and provides a value corresponding to the Calculated GRV.

The mean calculated gastric residual volume was shown to be significantly higher for patients in Group 2 than for those in Group 1 (109±11 versus 72±10 mL, respectively, p<0.05). Using the final pre-diluted BV (i.e., the Brix value before water dilution) to derive the % concentration of the dietary formula, in combination with the calculated gastric residual volume (Calc. GRV), the specific volume of dietary formula of dietary present at 180 minutes was determined for both groups. The volume of dietary formula remaining was significantly higher for patients in Group 2 compared to those in Group 1 (55±3 versus 6.8±1.2 mL, respectively, p<0.05).

The measured and analyzed results, use of refractometry, in this embodiment, in combination with conventional calculation of gastric residual volume identified 4% (1/25) of patients in Group 1 with low gastric residual volumes who might have possible gastric dysmotility (>20% of the initial 250 mL bolus volume of formula remaining at 180 minutes). Use of refractometry together with conventional measurement of gastric residual volumes indicated that 72% (13/18) of patients in Group 2 with higher gastric residual volumes had sufficient gastric emptying (<20% of initial 250 mL volume of formula remaining).

The full strength dietary formula is rich in dissolved nutrients and displays a high BV of 22.7±0.8. By contrast, saliva and gastric secretions have few dissolved substances and therefore have correspondingly low BVs, close to that of 0.9% sodium chloride. By evaluating the BV value, one may determine whether gastric contents are comprised predominately of dietary formula or digestive secretions in patients receiving dietary formula.

Monitoring GRV and Dietary Formula Concentration in Determining Feeding Tolerance

Brix value measurements and monitoring of GRVs and dietary formula concentrations are useful in evaluating feeding tolerance and gastric emptying. Embodying monitoring methods are exemplified in Table 2. A patient carrying a low GRV associated with low food retention (low Brix reading) would be interpreted to indicate that the formula is being emptied appropriately, and that there is no retention within the stomach. The risk for aspiration in the presence of low GRVs would be expected to be minimal, and the patient would be perceived as tolerating feeds. On the other hand, high GRV associated with high food retention would indicate the presence of delayed gastric emptying, an increased volume of gastric contents as a result of formula being retained in the stomach, and true feeding intolerance. However, patients carrying a low GRV but showing evidence of high food retention might be interpreted as having evidence of gastric dysmotility. In that case, the low GRV might represent a false negative screening monitor caused by the fact that the trigger of the feeding tube is not in the pool of gastric juice, or the trigger of feeding tube is adherent to the gastric mucosa. Low food retention by several BV determinations in a patient with high GRVs, may give some assurance that formula is being emptied effectively from the stomach. The high GRVs would thus represent a false positive screening monitor. Feeds could be continued as close clinical assessment is continued.

TABLE 2
Monitoring food content and residual volume simultaneously (prior to next feeding) in
patients receiving bolus nasogastric feeding.

| | Formula | | |
|---|---|---|---|
| Residual | Concentration (Brix | | Recommendation for |
| volume (GRV) | Value) | Interpretation | clinical practice |
| Low | Low | Good gastric emptying | Continue or even |
| | | | increase the tube |
| | | | feeding |
| High | High | Delayed gastric | Stop or decrease the |
| | | emptying | tube feeding, change |
| | | | from bolus-to continuous |
| | | | feeding, and/or switch to |
| | | | small bowel tube feeding |
| Low | High | May represent gastric | Check the true residual |
| | | dysmotility; aspirated | volume by water dilution |
| | | GRV may be | technique, close clinical |
| | | insensitive to true GRV | monitoring to assure |
| | | | tolerance |
| High | Low | May represent normal | Feeds could be |
| | | emptying of formula; | continued with close |
| | | elevated aspirated GRV | assessment of tolerance |
| | | may reflect volume of | to enteral feeding; |
| | | endogenous secretions | consider trial of anti- |
| | | | secretory agents such as |
| | | | proton pump inhibitor |

### Example 3

Continuous Nasogastric Tube Feeding: Monitoring by Brix Value and Conventional Gastric Residual Volumes

Materials and Methods

After monitoring for 24 hours, 36 patients on continuous enteral tube feeding with a full strength (100%) polymeric dietary formula (Osmolite HN) were entered in this study and divided into 2 groups based on their pattern of conventional aspirated gastric residual volumes over the monitoring period. Patients with lower aspirated gastric residual volumes (<75 mL) were placed in Group 1, while patients with higher aspirated gastric residual volumes (>75 mL on at least 2 occasions) were placed in Group 2. Aspirated gastric residual volumes were obtained by aspiration of the feeding tube using a 60-ml syringe, first in the supine position, and then in the right lateral decubitus position. Upon entry, all gastric contents were aspirated, the volume recorded (aspirated gastric residual volume), Brix value measurements by refractometry performed, and the contents reinstilled but diluted with 30 mL additional water. Then small amount was reaspirated, and repeat Brix value measurements were made. Three hours later, the entire procedure was repeated a second time.

The Brix values were measured using the device or system according to this invention, whose Brix scale of 0-32 could be read in 0.2 increments. The device or system was calibrated with distilled water before each measurement. One or two drops of the specimen fluid were placed on a designated window for observation using daylight at room temperature. In this way, the concentration of soluble solids in solution was measured at the bedside for each specimen.

Results

Results are presented as mean values±SEM. These results were close to the inventor's previous invention as disclosed in the pending application Ser. No. 10/787,705, using a conventional refractometer (N.O.W. 507-1, Nippon Optical Works; Tokyo, Japan). The Student's t test was used to assess differences in results between patients in Group 1 and Group 2. A p value of less than 0.05 was considered to be statistically significant.

No patient in either group demonstrated nausea, vomiting, aspiration, or evidence of clear intolerance of enteral tube feeding. Patients in Group 1, with lower Asp GRVs based on the pre-study period of monitoring, continued to demonstrate very low Asp GRVs following entry into the study, with 93% (43/44) of the Asp GRVs obtained on the first and second measurements being <75 ml. In contrast, patients in Group 2 (again differentiated by higher GRVs on pre-study monitoring) continued to show higher Asp GRVs following entry into the study, with only 11% (3/28) of Asp GRVs on both measurements <75 ml. Mean aspirated GRV was significantly higher for those patients in Group 2 compared to those in Group 1 on both first (130±3 versus 15+3 ml, respectively, p<0.05) and second (80±8 versus 11+8 ml, respectively, p<0.05) measurements.

The first and second Brix value measurements pre- and post-dilution were conducted. In general, patients in Group 1 tended to show of pattern of high pre-dilution Brix values dropping further to lower post-dilution Brix values than those patients in Group 2 (which showed the opposite pattern low pre-dilution Brix values dropping to a less extent to higher post-dilution Brix values). Only the difference in post-dilution Brix values between the two groups on both measurements reached statistical significance.

This pattern in Group 1 patients suggested that gastric contents were comprised of enteral formula of fairly high concentration (as evidenced by pre-dilution Brix values close to the in vitro Brix values range of 23.1 to 23.6 for full strength Osmolite HN) that was of very small volume (as evidenced by the tremendous drop in Brix values with dilution by a small 30 ml volume of distilled water). The opposite pattern in Group 2 patients, in contrast, suggested greater dilution by endogenous secretions (as evidenced by pre-dilution Brix values less than the Brix value range of 23.1 to 23.6 for full strength Osmolite HIS and greater total volume (as evidenced by the lesser drop in Brix values with dilution by a small 30 ml volume of distilled water).

The mean Brix value ratios for the two groups reflected these distinct patterns. The mean Brix value ratio was significantly higher for those patients in Group 2 compared to those in Group 1 on both the first (75±6% versus 22±2%, respectively, p<0.05) and second (62±4% versus 35±2%, respectively, p<0.05) measurements. For those patients in Group 1, Brix value ratios on all measurements for all patients were <70%. For those patients in Group 2, the Brix value ratios on the first measurement were >70% on all but one patient (patient #5). When measured the second time, 6 of the 14 patients (patients #1, 4-8) showed that the Brix value ratios had fallen to <70%.

As a whole, patients in Group 1 had a volume of formula estimated to be remaining on both measurements that was very low, reinforcing the pattern shown by Brix value ratios. In one patient (patient #22) where the volume of formula was greater than the hourly feeding rate, aspirated GRV and calculated GRV were <40 ml. Overall, 95% (21/22) of the Group 1 patients had a volume of formula remaining on both measurements that was less than the hourly feeding rate.

In contrast to those patients in Group 1, patients in Group 2 showed evidence of reduced gastric emptying and greater volume of retained formula. Comparing the formula estimated to be remaining with the hourly infusion rate, six patients is (patients #9-14) showed concurrence on both measurements for evidence of retention of formula and decreased or impaired gastric emptying. For these six patients, the volume of formula remaining was estimated to be greater than the hourly infusion rate on both measurements. In these six, both the first and second Brix value ratios were >70%. Traditional Asp GRV, however, was insensitive and failed to identify these patients, being 100 ml on two of the six first round of measurements, and six out of six on the second round of measurements. Three patients from Group 2 (patients #6-8), who on initial measurement showed retention of formula, improved on follow-up measurement indicating adequate gastric emptying of the formula. Five patients could be identified (patients #1-5) with high Asp GRVs from Group 2 who had a volume of formula remaining estimated to be less than the hourly infusion rate, assuring adequate gastric emptying all along.

As noted elsewhere in this application, a Brix value dilution test may be performed by measuring the Brix value of a gastric content sample to obtain a pre-dilution Brix value, adding a known volume of water to the gastric content sample to form a post-dilution sample, infusing the post-dilution sample into the stomach of a subject (patient), aspirating the post-dilution sample from the stomach, measuring the Brix value of the aspirated post-dilution sample to obtain a post-dilution Brix value, and dividing the post-dilution Brix value by the pre-dilution Brix value to obtain a Brix value ratio.

In one embodiment, the Brix value ratio is less than 70%, as this study has shown, there is a low likelihood for retention of formula and feeds may continue. In another embodiment, if the Brix value ratio is greater than 70% on for example, more than 2 occasions (e.g., performed every 4 hours), the clinician calculates the volume of dietary formula remaining in the stomach. The number of times that the Brix value ratio is greater than 70% and the time interval between performing successive tests is dependent on the clinical setting of the patient and easily determined by the ordinary practitioner. The finding that the volume of dietary formula remaining in the stomach is greater than the hourly infusion rate alerts the clinician to the possibility of delayed gastric emptying with retention of formula, and that feeds may be continued with caution under close observation (for example, continuing to perform Brix value dilution tests with aspirated gastric residual volumes every 4 hours or some other determined time period).

### Example 4

Determination of Gastric Residual Volume (GRV)

Materials and Methods

Forty-three patients receiving bolus nasogastric feeding were monitored for 24 hours prior to entry into the study, and then divided into two groups based on conventional use of GRV; patients with low GRVs (<75 ml) were placed in Group 1, while patients with higher GRVs (>75 ml) were placed in group 2. All subjects were given 250 ml of polymeric formula by bolus nasogastric infusion, followed by Brix value (BV) measurement of gastric contents at 0, 30, 60, 120, and 180 minutes. All gastric fluid was aspirated after 180 minutes of feeding; the volume was recorded (Aspirate GRV) and BV made (PreDilute BV), then reinstilled with an added 30 ml of dilutional water, after which a final aspiration and BV measurement (PostDilute BV) was performed. Calculated GRV was determined by the equation: Calculated GRV times PostDilute BV=(Calculated GRV+30 ml) times PostDilute BV. Specific volume of formula at 180 minutes was defined by 2 equations; % Concentration=BV180 min/0.24 and Volumeformula=% Concentration times Calculated GRV.

Results

Serial BV measurements decreased in both groups after bolus feeding. These results were close to the inventor's previous invention as disclosed in the pending application Ser. No. 10/787,705, using a conventional refractometer (N.O.W. 507-1, Nippon Optical Works; Tokyo, Japan). For patients in Group 2 the decrease was less, such that at 180 minutes the mean BV for gastric contents was, significantly higher than for those patients in Group 1 (10.5 versus 5.8, respectively, p<0.01). Aspirate GRV, Calculated GRV, and Volume.sub.formula present at 180 minutes was significantly greater for patients in Group 2 compared to those in Group 1. Use of refractometry in combination with traditional use of GRV identified 4% (1/25) of patients in Group 1 with low GRVs who might have possible gastric dysmotility (>20% of initial 250 ml volume of formula remaining at 180 minutes), and assured that 72% (13/18) patients in Group 2 with higher GRVs had sufficient gastric emptying (<20% of initial 250 ml volume of formula remaining).

### Example 5

Determination of Gastric Residual Volume (GRV) by Gastrometer®

A medical device, Gastrometer® is manufactured as one embodiment according to this invention, with the detecting sample means 20 by using refractometry or photodynamics, preferably, using refractometry to determine gastric residual volume. A system as FIG. 3(B) shown could be designed comprising Gastrometer®, such that the system is capable for conducting and determining the intestinal parameters according to this invention. After receiving a gastric content sample, using Gastrometer® to measure the Brix value of a gastric content sample to obtain a pre-dilution Brix value; adding a known volume of water to the gastric content sample to form a post-dilution sample, infusing the post-dilution sample into the stomach of a subject, and aspirating the post-dilution sample. Aspirated post-dilution sample was then obtained, using Gastrometer® to measure the Brix value of the aspirated post-dilution sample to have a post-dilution Brix value, and using Gastrometer® to calculate by multiplying the Brix value of the gastric content sample by the known volume of water and dividing the resulting product by the difference between pre-dilution Brix value and the post-dilution value. In preferred embodiment, the equation of the multiplying step is set and saved in the reference database 21, and provided to be determined by the user through the keypad 14, and responding to its instruction shown on the display 13. The table below refers to using Gastrometer® to calculate GRVs in vitro related to different formulas, including Resource®-peach, and Resource®-strawberry (both from Novartis, USA).

Using Gastrometer® to Calculate GRVs in vitro using different formulas

| Formula | Calculated volume (ml) | | |
|---|---|---|---|
| Resource-peach | 198.5 | 326.2 | 482.1 |
| Resource-strawberry | 196.4 | 320.0 | 503.1 |
| Real volume (ml) | 200.0 | 350.0 | 500.0 |
| Real % conc. dietary formula | 50% | 57% | 60% |

Although the invention has been described with respect to particular embodiments, it will be apparent to those skilled in the art that various changes and modifications can be made without departing from the spirit and scope of invention, and it should be understood that this invention is not to be unduly limited to the illustrative embodiments set forth herein.

1. A test strip for use in conjunction with a medical device, wherein the medical device comprises a sample interface for measuring a property of a fluid sample, the test strip comprising:
a support member having a top surface and a bottom surface and an aperture suitable for receiving the fluid sample;
and wherein the test strip is configured to engage with the medical device such that the aperture of the test strip aligns with a sample interface of the medical device, thereby allowing the property of the fluid sample to be measured.

2. The test strip of claim 1, wherein the aperture extends from the top surface to the bottom surface of the support member.

3. The test strip of claim 1, wherein the aperture has an inner surface and wherein the inner surface is substantially non-reflective.

4. The test strip of claim 1, wherein the test strip is elongate and wherein at least a portion of the test strip is adapted to slide into a channel in the medical device.

5. The test strip of claim 1, wherein the volume of the fluid sample that can be received within the aperture is at least about 0.01 ml, preferably about 0.1 to about 10.0 ml and more preferably about 0.4 to about 2.0 ml.

6. The test strip of claim 1, which further comprises a system for removing fluid from the surface of the sample interface of the medical device when the test strip is withdrawn from the medical device.

7. The test strip of claim 1, wherein the support member has a distal end and a proximal end; and wherein the bottom surface of the support member is provided with a wiping system, preferably in the form of at least one resilient pad for removing fluid from the surface of the sample interface of the medical device when the test strip is withdrawn from the medical device.

8. A test strip for use in conjunction with a medical device, wherein the medical device comprises a sample interface for measuring a property of a fluid sample, the test strip comprising:
a support member having a distal end and a proximal end, a top surface and a bottom surface, and being provided with an aperture suitable for receiving a fluid sample extending from the top surface to the bottom surface, and a wiping system on the bottom surface of the support member for removing fluid from the surface of the sample interface of the medical device when the test strip is withdrawn from the medical device;
and wherein the test strip is configured to slidingly engage with the medical device such that the aperture of the test strip aligns with a sample interface of the medical device thereby allowing the property of the fluid sample to be measured.

9. The test strip of claim 8, wherein the wiping system comprises at least one resilient pad; and wherein correct engagement of the test strip with the medical device causes the at least one resilient pad to be compressed against an opposing surface of the medical device, such that on withdrawing the test strip from the medical device the resilient pad is drawn across the surface of the sample interface.

10. The test strip of claim 1, wherein a sealing system is provided about the aperture on the bottom surface of the support member such that when the test strip is correctly engaged with the medical device a substantially water-tight seal is formed between the aperture and the sample interface.

11. The test strip of claim 1, wherein a resilient material is disposed on the bottom surface of the support member surrounding the aperture such that when the test strip is correctly engaged with the medical device a substantially water-tight seal is formed between the aperture and the sample interface.

12. The test strip of claim 1, wherein the support member has a distal end and a proximal end; and wherein the proximal end is provided with a handle member for holding the test strip.

13. The test strip of claim 1, wherein the support member has a distal end and a proximal end; and wherein the proximal end is provided with a handle member for holding the test strip; and wherein the handle member has a textured or contoured surface for improving grip.

14. The test strip of claim 1, which further comprises a sleeve adapted to enclose at least a portion of the support member; wherein the sleeve comprises at least a lower section having upwardly extending lateral flanges which engage with the support member and a window at least as large as the aperture; and wherein the test strip is configured to engage with the medical device such that the window in the lower section of the sleeve aligns with the sample interface of the medical device and the aperture, such that there is a direct optical path from the sample interface through the window to the aperture.

15. The test strip of claim 1, which further comprises a sleeve adapted to enclose at least a portion of the support member; wherein the sleeve comprises at least a lower section having upwardly extending lateral flanges which are capable of engaging with and acting as guide members for the support member, and a window at least as large as the aperture; and wherein the support member is slidable within the sleeve from a first position in which the aperture is aligned with the window in the lower of the sleeve, to a second position in which the aperture is not aligned with the window and is enclosed by the lower section of the sleeve.

16. A test strip for use in conjunction with a medical device, wherein the medical device comprises a sample interface for measuring a property of a fluid sample, the test strip comprising:
an elongate support member having a distal end and a proximal end along its longitudinal axis, a top surface and a bottom surface, and being provided with an aperture suitable for receiving a fluid sample extending from the top surface to the bottom surface; and
a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section;
wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve;
and wherein the test strip is configured to engage with the medical device such that the window in the lower section of the sleeve aligns with the sample interface of the medical device and the aperture, such that there is a direct optical path from the sample interface through the window to the aperture, thereby allowing the property of the fluid sample to be measured.

17. The test strip of claim 16, which further comprises a wiping system for removing fluid from the surface of the sample interface of the medical device when the test strip is withdrawn from the medical device.

18. The test strip of claim 16, wherein the bottom surface of the support member is provided with at least one resilient pad; and wherein the sliding of the support member from the first position to the second position, while the test strip is correctly engaged with the medical device, causes the at least one resilient pad to be compressed against and drawn across at least the surface of the sample interface of the medical device.

19. The test strip of claim 16, wherein the lower section of the sleeve further comprises an upwardly extending distal flange.

20. The test strip of claim 1, which further comprises a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section;
wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve;
wherein the support member and sleeve are provided with a system for mutual engagement which engages when the support member is in the second position, thereby inhibiting free movement of the support member relative to the sleeve.

21. The test strip of claim 1, further comprising a lid for covering at least a part of the aperture in the top surface of the support member.

22. The test strip of claim 1, wherein the support member comprises two apertures; and wherein the test strip is configured to engage with the medical device such that each of the two apertures can be brought into alignment with a sample interface of the medical device.

23. The test strip of claim 1, wherein the support member has a distal end and a proximal end; and wherein the distal end is provided with a trigger; and wherein the trigger is configured to insert into a corresponding socket of the medical device and is adapted to communicate with the medical device.

24. The test strip of claim 1, wherein the support member has a distal end and a proximal end; and wherein the distal end of the is provided with a trigger; wherein the trigger is configured to insert into a corresponding socket of the medical device and is adapted to initiate a photo-interrupt.

25. The test strip of claim 16, wherein the lower section of the sleeve further comprises an upwardly extending distal flange; wherein the upwardly extending distal flange is provided with a trigger; and wherein the trigger is configured to insert into a corresponding socket of the medical device and is adapted to communicate with the medical device.

26. The test strip of claim 1 which is disposable.

27. The test strip of claim 16 which is disposable.

28. A test strip for use in conjunction with a medical device, wherein the medical device comprises a sample interface for measuring a property of a fluid sample, the test strip comprising;
an elongate support member having a distal end and a proximal end along its longitudinal axis, a top surface and a bottom surface, and being provided with an aperture suitable for receiving a fluid sample extending from the top surface to the bottom surface; and
a trigger which is configured to insert into a corresponding socket of the medical device and is adapted to communicate with the medical device;
at least one resilient pad on the bottom surface of the support member; and
a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section;
wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve;
wherein the test strip is configured to engage with the medical device such that the window in the lower section of the sleeve aligns with the sample interface of the medical device and the aperture, such that when the support member is in the first position there is a direct optical path from the sample interface through the window to the aperture, thereby allowing the property of the fluid sample to be measured;
wherein the sliding of the support member from the first position to the second position, while the test strip is correctly engaged with the medical device, causes the at least one resilient pad to be compressed against and drawn across at least the surface of the sample interface of the medical device; and
wherein the support member and sleeve are provided with a system for mutual engagement which engages when the support member is in the second position, thereby inhibiting free movement of the support member relative to the sleeve.

29. A test strip for use in conjunction with a medical device, wherein the medical device comprises a sample interface for measuring a property of a fluid sample, the test strip comprising:
an elongate support member having a distal end and a proximal end along its longitudinal axis, a top surface and a bottom surface, and being provided with an aperture suitable for receiving a fluid sample extending from the top surface to the bottom surface;
a handle member at the proximal end of the support member for holding the test strip;
a sealing system disposed about the aperture on the bottom surface of the support member, such that when the test strip is correctly engaged with the medical device a substantially water-tight seal is formed between the aperture and the sample interface;
a trigger which is configured to insert into a corresponding socket of the medical device and is adapted to communicate with the medical device;
at least one resilient pad on the bottom surface of the support member;
a lid for covering at least a part of the aperture in the top surface of the support member; and
a sleeve adapted to enclose at least a portion of the support member; wherein the sleeve comprises a lower section having upwardly extending lateral flanges which are capable of engaging with and acting as guide members for the support member, and a window at least as large as the aperture; and an upper section having an window, the upper section being engageable with the lower section of the sleeve such that the window in the upper section aligns with the window in the lower section;
wherein the support member is slidable within the sleeve from a first position in which the aperture is aligned with the windows in the lower and upper sections of the sleeve, to a second position in which the aperture is not aligned with the windows and is enclosed by the lower and upper sections of the sleeve;
wherein the test strip is configured to engage with the medical device such that the window in the lower section of the sleeve aligns with the sample interface of the medical device and the aperture, such that when the support member is in the first position there is a direct optical path from the sample interface through the window to the aperture, thereby allowing the property of the fluid sample to be measured;
wherein the sliding of the support member from the first position to the second position, while the test strip is correctly engaged with the medical device, causes the at least one resilient pad to be compressed against and drawn across at least the surface of the sample interface of the medical device; and
wherein the support member and sleeve are provided with a system for mutual engagement which engages when the support member is in the second position, thereby inhibiting free movement of the support member relative to the sleeve.

30. A medical system for measuring a property of a fluid sample, comprising:
a medical device capable of measuring at least one property of the fluid sample and having a sample interface for receiving a fluid sample to be measured; and a test strip;
wherein the test strip comprises:
a support member having a top surface and a bottom surface and an aperture suitable for receiving the fluid sample;
and wherein the medical device is provided with a channel configured to receive at least a portion of the test strip, and the test strip is configured to engage with the medical device such that the aperture of the test strip aligns with a sample interface of the medical device, thereby allowing the property of the fluid sample to be measured.

31. A medical system for measuring a property of a fluid sample, comprising:
a medical device capable of measuring at least one property of the fluid sample and having a sample interface for receiving a fluid sample to be measured; and a test strip; wherein the test strip comprises:
a support member having a distal end and a proximal end, a top surface and a bottom surface, and being provided with an aperture suitable for receiving a fluid sample extending from the top surface to the bottom surface, and a wiping system on the bottom surface of the support member for removing fluid from the surface of the sample interface of the medical device when the test strip is withdrawn from the medical device;
and wherein the medical device is provided with a channel configured to receive at least a portion of the test strip, and the test strip is configured to slidingly engage with the channel of the medical device such that the aperture of the test strip aligns with a sample interface of the medical device, thereby allowing the property of the fluid sample to be measured.

32. The medical system of claim 30, wherein the medical device is a refractometer which measures at least the refractive index of a sample of fluid.

33. A medical system of claim 30, wherein the medical device further comprises a digital display for reporting a measurement of a property of the fluid sample.

34. A medical system of claim 30, wherein the medical device further comprises a keypad for manual communication with the medical device.

35. The medical system of claim 30, which further comprises a docking station, the docking station capable of electronic communication with the medical device for downloading and/or uploading data electronically.

36. The medical system of claim 30, wherein the medical device is a refractometer which measures at least the refractive index of a sample of fluid; and wherein the refractometer converts a measurement of a refractive index of a fluid into a different property of the of the fluid.

37. The medical system of claim 30, wherein the medical device is a refractometer which measures at least the refractive index of a sample of fluid; and wherein the refractometer converts a measurement of a refractive index of a fluid into a measurement of the Brix value (BV) of the fluid.

38. The medical system of claim 30, wherein the medical device is a refractometer, and wherein the refractometer comprises means for calculating the gastric residual volume (GRV) in a subject based on the measured Brix values (BV) of at least two samples of gastric fluid, at least one of which samples was obtained from the subject after the gastric contents had been diluted in situ with a known volume of a fluid, and at least one of which samples was obtained from the subject before the gastric contents had been diluted with the known volume of a fluid;
wherein the means for calculating the GRV in the subject calculates the GRV using the equation:
GRV=(BV2×Vol)/(BV1−BV2)
wherein BV1 is the measured pre-dilution BV of a sample of gastric fluid before dilution of gastric contents with a known volume of fluid (Vol), and BV2 is the measured post-dilution BV of a sample of gastric fluid after dilution of gastric contents with the known volume of fluid (Vol).

39. An optical module for a refractometer, comprising:
a prism having a sample interface for contacting a fluid sample to be measured;
a light source arranged to project light through a first face of the prism onto the sample surface; and
an array of photoreceptors arranged proximal to a second face of the prism to detect light reflected from the sample interface when a fluid sample is in contact with the sample interface;
wherein the light source is a laser diode; and
wherein the array of photoreceptors comprises one or more photodiodes.

40. An optical module according to claim 39, wherein the wavelength of the laser is from about 600 to about 1300 nm, preferably from about 650 to about 850 nm and more preferably from about 700 to about 800 nm.

41. An optical module according to claim 39, wherein the prism is made from a material selected from the group consisting of glass, quartz and plastic.

42. An optical module according to claim 39, wherein the laser diode projects light onto the sample interface at an angle of incidence of about 42 to about 62°, preferably from about 50 to about 62° and more preferably from about 55 to about 62°.

43. An optical module according to claim 39, wherein the array of photoreceptors comprises two photodiodes.

44. An optical module for a refractometer, comprising:
a prism having sample interface for contacting a fluid sample to be measured;
a light source arranged to project light through a first face of the prism onto the sample surface; and
an array of photoreceptors arranged proximal to a second face of the prism to detect light reflected from the sample interface when a fluid sample is in contact with the interface;
wherein the light source is a laser diode that is arranged to project light onto the sample interface at an angle of incidence of about 42 to about 62°, preferably from about 50 to about 62° and more preferably from about 55 to about 62°; wherein the wavelength of the laser is from about 600 to about 1300 nm, preferably from about 650 to about 850 nm and more preferably from about 700 to about 800 nm; and
wherein the array of photoreceptors comprises one or more photodiodes.
